(19) United States
(12) Patent Application Publication
(10) Pub. No.: US 2015/0265525 A1
(43) Pub. Date: Sep. 24, 2015

(54) COMPOSITION FOR ALTERING THE COLOR OF KERATIN FIBERS

(71) Applicant: L'OREAL, Paris (FR)

(72) Inventors: Mark BENN, Union, NJ (US); Michael DEGEORGE, Old Bridge, NJ (US)

(73) Assignee: L'OREAL, Paris (FR)

(21) Appl. No.: 14/221,875

(22) Filed: Mar. 21, 2014

(51) Int. Cl. A61K 8/92; B65D 81/32; A61K 8/41; A61Q 5/10; A61K 8/81

(52) U.S. Cl. CPC A61K 8/92; A61Q 5/10; A61K 8/8147; A61K 8/416; B65D 81/32

(57) ABSTRACT

Disclosed herein is a cosmetic composition for altering the color of hair containing a fatty substance; an acrylic polymer selected from crosslinked copolymers of (meth)acrylic acid and/or (C1-C6)alkyl esters and from acrylic associative polymers; a salt selected from an ammonium salt other than an ammonium acetate salt, a quaternary ammonium salt, a quaternary diammonium salt, an alkaline earth metal salt, a transition metal salt, an alkali metal salt other than an alkali metal phosphate salt, an agmatine salt, and mixtures thereof; and a cosmetically acceptable solvent; and optionally, a colorant compound. Also disclosed is a process for altering the color of hair and depositing color onto hair wherein a composition comprising the cosmetic composition and an oxidizing composition containing an oxidizing agent is applied onto hair.

### FIELD OF THE INVENTION

The present invention relates to a cosmetic composition for altering the color of keratin fibers such as the hair. The cosmetic composition comprises at least one fatty substance, at least one acrylic polymer, at least one salt and a cosmetically acceptable solvent. The cosmetic composition further comprises at least one oxidizing agent and may additionally contain a colorant compound.

### BACKGROUND OF THE INVENTION

It is known that consumers desire to use cosmetic and personal care compositions that enhance the appearance of keratin fibers such as hair by changing the color of the hair and/or by imparting various properties to hair, for example, shine and conditioning. The process of changing the color of hair can involve depositing an artificial color onto the hair which provides a different shade or color to the hair and/or lifting the color of the hair, such as lightening the color of dark hair to lighter shades.

The process of lifting the color of hair, also known as lightening, generally requires the use of compositions that comprise at least one oxidizing agent. When colorants or dye compounds such as oxidation dye precursors and direct dyes are present in these compositions, such compositions can change or deposit color and lighten the color of hair at the same time. Conventional hair coloring products are permanent dye compositions comprising oxidation dye precursors, which are also known as primary intermediates or couplers. These oxidation dye precursors are colorless or weakly colored compounds which, when combined with oxidizing agents, give rise to colored complexes by a process of oxidative condensation.

In general, hair lightening or color lifting compositions and hair dyeing compositions possess an alkalinity such that these compositions have a pH value of above 7, typically being at pH 9 and above, and may generally require the presence of an alkalizing agent such as ammonia or an ammonia gas generating compound and/or an amine or ammonium-based compound in amounts sufficient to make such compositions alkaline. The alkalizing agent causes the hair shaft to swell, thus allowing the small oxidative dye molecules to penetrate the cuticle and cortex before the oxidation condensation process is completed. The resulting larger-sized colored complexes from the oxidative reaction are then trapped inside the hair fiber, thereby permanently altering the color of the hair. While such hair dyeing and/or color lifting compositions can effectively alter the color of hair, these compositions can damage the hair fibers and/or irritate the scalp and may be accompanied by an undesirable odor of ammonia.

Thus, in order to reduce or avoid the drawbacks above, as well as to improve the cosmetic performance of hair color lifting and hair dyeing compositions, the use of new and additional ingredients and novel combinations of ingredients are continuously sought. However, the choice of ingredients or combinations of ingredients could pose difficulties insofar as they cannot be detrimental to other cosmetic attributes such as ease and uniformity of application, rheology or viscosity properties and stability of the compositions, color deposit and target shade formation, and/or result into more disadvantages such as increased damage or a less healthy look to the hair. It is therefore, desirable to provide the consumer with compositions and methods that can lift the color of hair and additionally, deposit color onto hair in an efficient or improved manner, while providing other cosmetic advantages such as shine, conditioning, and a healthy appearance to the hair. Furthermore, it is preferable to formulate such compositions that are less costly to manufacture by requiring less ingredients and/or lower levels of ingredients and/or employing a more efficient process of manufacture.

Thus, the objective of the present invention is to obtain novel compositions for altering the color of hair by lifting or lightening the color of the hair and additionally, depositing color while providing conditioning, a healthy and shiny appearance to hair and minimizing the damage to the hair and other adverse effects to the consumer. Another objective of the invention is to provide compositions that have a unique, non-drip consistency or rheology and yet spreads easily on the hair.

### BRIEF SUMMARY OF THE INVENTION

The present disclosure is directed to compositions and methods for changing or altering the color of hair. Exemplary methods comprise applying a composition comprising a cosmetic composition and an oxidizing composition, and subsequently applying the composition to the hair in order to lift or lighten the color of the hair. When the composition also contains dye compounds, the composition is also able to deposit color onto the hair.

By way of example, there is a need to provide compositions and processes that provide for hair to be lightened, and if desired, additionally colored, while achieving a desired change in hair tone and minimizing damage to the hair. Lightening or lifting the color of the hair is typically evaluated by the variation in tone height before and after the application of a hair color-altering composition onto hair. This variation corresponds to the degree or level of lightening or lift. The notion of “tone” is based on the classification of the natural shades, one tone separating each shade from the shade immediately following or preceding it, which is well known to hairstyling professionals. The tone heights or levels range from 1 (black) to 10 (light blond), one unit corresponding to one tone; thus, the higher the number, the lighter the shade or the greater the degree of lift.

Accordingly, in various exemplary embodiments of the disclosure, the compositions and methods described allow one to achieve a desired level of color “lift” in tone, i.e. to a higher number. In further exemplary embodiments, the compositions and methods described below allow one to additionally deposit color onto hair.

In order to achieve these and other advantages, the present invention is drawn to a cosmetic composition comprising:
(a) from about 10% to about 80% by weight of at least one fatty substance;
(b) at least one acrylic polymer selected from crosslinked copolymers of (meth)acrylic acid and/or (C1-C6)alkyl esters and from acrylic associative polymers;
(c) from about 0.5% to about 15% by weight of at least one salt selected from an ammonium salt other than an ammonium acetate salt, a quaternary ammonium salt, a quaternary diammonium salt, an alkaline earth metal salt, a transition metal salt, an alkali metal salt other than an alkali metal phosphate salt, an agmatine salt, and mixtures thereof; and
(d) at least one cosmetically acceptable solvent;
   all weights being based on the total weight of the composition.

The present invention is also drawn to compositions comprising the above-described cosmetic composition and an oxidizing composition containing at least one oxidizing agent and a cosmetically acceptable solvent selected from water and a water/organic solvent mixture. The present invention is also drawn to a process of lifting or lightening the color of keratin fibers, such as hair, comprising applying onto the said fibers, a composition comprising the above-described cosmetic and oxidizing compositions; and leaving the composition on the fibers for a period of time sufficient to lighten the fibers. The above-described cosmetic composition may further comprise at least one colorant compound.

### DETAILED DESCRIPTION OF THE INVENTION

Other than in the operating examples, or where otherwise indicated, all numbers expressing quantities of ingredients and/or reaction conditions are to be understood as being modified in all instances by the term “about” which encompasses +10%.

“At least one” as used herein means one or more and thus includes individual components as well as mixtures/combinations.

“Keratin fiber” may be chosen from, for example, human hair.

The term “altering the color” or “color-altering” as used herein may refer lifting or lightening the color of hair. It can also refer to dyeing or coloring hair or depositing color onto the hair. In certain instances, it refers to lifting or lightening the color of hair and depositing color onto the hair at the same time.

“Formed from,” as used herein, means obtained from chemical reaction of, wherein “chemical reaction,” includes spontaneous chemical reactions and induced chemical reactions. As used herein, the phrase “formed from”, is open ended and does not limit the components of the composition to those listed, e.g., as component (i) and component (ii). Furthermore, the phrase “formed from” does not limit the order of adding components to the composition or require that the listed components (e.g., components (i) and (ii)) be added to the composition before any other components.

“Hydrocarbons,” as used herein, include alkanes, alkenes, and alkynes, wherein the alkanes comprise at least one carbon, and the alkenes and alkynes each comprise at least two carbons; further wherein the hydrocarbons may be chosen from linear hydrocarbons, branched hydrocarbons, and cyclic hydrocarbons; further wherein the hydrocarbons may optionally be substituted; and further wherein the hydrocarbons may optionally further comprise at least one heteroatom intercalated in the hydrocarbon chain.

“Silicone compound,” as used herein, includes, for example, silica, silanes, silazanes, siloxanes, and organosiloxanes; and refers to a compound comprising at least one silicon; wherein the silicone compound may be chosen from linear silicone compounds, branched silicone compounds, and cyclic silicone compounds; further wherein the silicone compound may optionally be substituted; and further wherein the silicone compound may optionally further comprise at least one heteroatom intercalated in the silicone chain, wherein the at least one heteroatom is different from the at least one silicon.

“Substituted,” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalkyl groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

“Polymers,” as defined herein, include homopolymers and copolymers formed from at least two different types of monomers.

“(Meth)acrylic” as used herein, is understood to mean, within the meaning of the present patent application, “acrylic or methacrylic”.

The term “substantially free of ammonia” as defined herein means that the compositions of the present invention are completely free of ammonia (including ammonium ions) or contains no appreciable amount of ammonia (including ammonium ions) or contains no appreciable amounts of ammonia gas generating compounds, for example, no more than 1% by weight, or no more than 0.5% by weight, or no more than 0.3% by weight, or no more than 0.1% by weight, based on the weight of the cosmetic compositions or compositions comprising the cosmetic composition and the oxidizing composition of the invention.

It has been surprisingly and unexpectedly discovered that when the cosmetic composition of the present invention contains an oxidizing agent or when it is mixed with an oxidizing composition containing an oxidizing agent, improved hair color lightening or lifting effects are achieved. For example, the composition provides good uniformity of lift along the fiber between the tip and the root of the hair (also called the selectivity of lightening). Moreover, the composition can be applied without difficulty onto keratin fibers without running or dripping and less amount of oxidizing agent can be used in order to achieve the desired level of lift or lightening.

It has also now been surprisingly discovered that altering the color of hair by way of lifting the color of hair can be achieved using the process and compositions of the present invention, and particularly, using a composition having a pH of from 2 to 7 and comprising the cosmetic composition and oxidizing composition of the present invention. Moreover, it was surprisingly and unexpectedly discovered that by using the compositions of the present invention, it was possible to achieve acceptable lift to the color of the hair that corresponds to an increase in tone height in an amount ranging from 0.5 to 4.

Furthermore, when the cosmetic composition of the present invention additionally contains dye compounds, it was surprisingly and unexpectedly discovered that the composition can also deposit color effectively and comparably to, if not better, than traditional or commercial hair dyes using similar or less amounts of dye compounds. Thus, the compositions and process of the present invention can provide for improved color visibility and better color coverage.

It has also been surprisingly and unexpectedly found that the cosmetic composition of the invention can provide the desired cosmetic performance and attributes at lower manufacturing costs due to one or more of the following factors: lower amounts of dyes used, nature or number of ingredients, less time of production, and process of making wherein the cosmetic composition of the present invention can be prepared by a cold process method, i.e., without the use of heat.

In an embodiment, the present invention relates to a cosmetic composition containing:
(a) from about 10% to about 80% by weight of at least one fatty substance;
(b) from about 0.1% to about 10% by weight of active material of at least one acrylic polymer selected from crosslinked copolymers of (meth)acrylic acid and/or (C1-C6)alkyl esters and from acrylic associative polymers;
(c) from about 0.5% to about 15% by weight of at least one salt selected from an ammonium salt other than an ammonium acetate salt, a quaternary ammonium salt, a quaternary diammonium salt, an alkaline earth metal salt, a transition metal salt, an alkali metal salt other than an alkali metal phosphate salt, an agmatine salt, and mixtures thereof; and
(d) at least one cosmetically acceptable solvent;
   all weights being based on the total weight of the composition.

In one embodiment, the present invention relates to a cosmetic composition containing:
(a) from about 30% to about 60% by weight of at least one fatty substance;
(b) from about 1% to about 3% by weight of active material of an acrylic polymer selected from an acrylates copolymer in an aqueous dispersion;
(c) from about 3% to about 8% by weight of at least one salt selected from ammonium chloride, ammonium sulfate, calcium chloride, manganese gluconate, sodium acetate, sodium sulfate, agmatine sulfate, and mixtures thereof;
(d) at least one cosmetically acceptable solvent;
   all weights being based on the total weight of the composition.

The above-described cosmetic compositions may further comprise one or more of at least one neutralizing agent, at least one colorant compound, at least one nonionic surfactant, auxiliary agents suitable for use in cosmetic compositions. The at least one colorant compound may be selected from oxidative dye precursors, direct dyes, pigments, and mixtures thereof.

The above-described cosmetic compositions are capable of being mixed with an oxidizing composition containing at least oxidizing agent selected from peroxides, urea peroxide, alkali metal bromates, ferricyanides, peroxygenated salts, perborates, percarbonates, laccases, peroxidases, redox enzymes, and mixtures thereof, and a cosmetically acceptable solvent selected from water and a water/organic solvent mixture. The resulting composition comprising the cosmetic composition and the oxidizing composition is used for lifting or lightening the composition of the hair. When the cosmetic composition additionally contains a colorant compound, the resulting composition is also used for depositing color onto hair.

In an embodiment, the present invention also relates to a composition for altering the color of hair containing:
A. a cosmetic composition containing:
   (a) from about 10% to about 80% by weight of at least one fatty substance;
   (b) from about 0.1% to about 10% by weight of active material of at least one acrylic polymer selected from crosslinked copolymers of (meth)acrylic acid and/or (C1-C6)alkyl esters and from acrylic associative polymers;
   (c) from about 0.5% to about 15% by weight of at least one salt selected from an ammonium salt other than an ammonium acetate salt, a quaternary ammonium salt, a quaternary diammonium salt, an alkaline earth metal salt, a transition metal salt, an alkali metal salt other than an alkali metal phosphate salt, an agmatine salt, and mixtures thereof; and
   (d) at least one cosmetically acceptable solvent;
      all weights being based on the total weight of the composition;
B. an oxidizing composition containing at least one oxidizing agent selected from peroxides, urea peroxide, alkali metal bromates, ferricyanides, peroxygenated salts, perborates, percarbonates, laccases, peroxidases, redox enzymes, and mixtures thereof, and a cosmetically acceptable solvent selected from water and a water/organic solvent mixture;
   wherein the pH of the composition for altering the color of hair ranges from about 2 to about 7.

The above-described composition for altering the color of hair may be employed to lift or lighten the color of hair.

The above-described cosmetic composition may further comprise one or more of at least one neutralizing agent, at least one colorant compound, at least one nonionic surfactant, auxiliary agents suitable for use in cosmetic compositions. The at least one colorant compound may be selected from oxidative dye precursors, direct dyes, pigments, and mixtures thereof. When the cosmetic composition additionally contains a colorant compound, the composition for altering the color of hair is also used for depositing color onto hair.

In another embodiment, the present invention also relates to a composition for altering the color of hair containing:
(a) from about 20% to about 60% by weight of at least one fatty substance;
(b) from about 0.5% to about 1.5% by weight of active material of an acrylic polymer selected from an acrylates copolymer in an aqueous dispersion;
(c) from about 1.5% to about 4% by weight of at least one salt selected from ammonium chloride, ammonium sulfate, calcium chloride, manganese gluconate, sodium acetate, sodium sulfate, agmatine sulfate, and mixtures thereof;
(d) at least one cosmetically acceptable solvent; and
(e) from about 0.01 to about 1% by weight of at least one neutralizing agent other than (c) and selected from alkali metal carbonates, alkali metal phosphates, organic amines, hydroxide base compounds, and mixtures thereof;
(f) at least one colorant compound selected from oxidative dye precursors, direct dyes, pigments, and mixtures thereof;
(g) at least one nonionic surfactant selected from alkoxylated nonionic surfactants; and
(h) at least one oxidizing agent;
   all weights being based on the total weight of the composition; and
   wherein the pH of the composition ranges from about 4 to about 6.9.

In preferred embodiments, the above-described compositions of the present invention are substantially free of ammonia or ammonia gas generating compounds.

According to another embodiment of the invention, a kit for altering the color of keratin fibers, such as hair, is provided, comprising a first unit containing any one of the above described cosmetic compositions and a second unit comprising the above described oxidizing composition.

According to other embodiments, a process for altering the color of keratin fibers, such as hair, is provided, comprising applying to the hair, a composition comprising any one of the above described cosmetic compositions and the above described oxidizing composition.

According some embodiments, the viscosity of any one of the above described cosmetic compositions is from about 40 uD to about 80 uD, or such as from about 60 uD to about 80 uD, or such as from about 70 uD to about 80 uD

According to yet other embodiments, the viscosity of the composition comprising any one of the above described cosmetic compositions and the above described oxidizing composition is from about 40 uD to about 80 uD, or such as from about 60 uD to about 80 uD, or such as from about 70 uD to about 80 uD.

Viscosity in uD (units of deflection) was measured by a Mettler RM 180 Rheomat, spindle #3 at 25° C.

### Fatty Substances

The cosmetic composition of the present invention comprises at least one fatty substance.

“Fatty substance” means an organic compound insoluble in water at normal temperature (25° C.) and at atmospheric pressure (750 mmHg) (solubility below 5% and such as below 1% and further such as below 0.1%). Fatty substances have in their structure a chain of at least two siloxane groups or at least one hydrocarbon chain having at least 6 carbon atoms. Moreover, fatty substances are generally soluble in organic solvents in the same conditions of temperature and pressure, for example in chloroform, ethanol, benzene or decamethylcyclopentasiloxane.

The method of measuring the viscosity of fatty substances such as oils and esters, can be any standard method known in the industry. Viscosity can be expressed as a kinematic viscosity or dynamic viscosity.

Fatty substances are, for example, chosen from alkanes, fatty alcohols, esters of fatty acid, esters of fatty alcohol, oils such as mineral, vegetable, animal and synthetic non-silicone oils, non-silicone waxes and silicones.

In some embodiments, the alcohols and esters have at least one linear or branched, saturated or unsaturated hydrocarbon group, comprising 6 to 30 carbon atoms, optionally substituted, for example, with at least one hydroxyl group (for example 1 to 4). If they are unsaturated, these compounds can have one to three, conjugated or unconjugated, carbon-carbon double bonds.

With regard to the alkanes, in some embodiments, these have from 6 to 16 carbon atoms and are linear or branched, optionally cyclic. As examples, alkanes can be chosen from hexane and dodecane, isoparaffins such as isohexadecane, isododecane, and isodecane.

Non-limiting examples of non-silicone oils usable in the composition of the disclosure, include: hydrocarbon oils of animal origin, such as perhydrosqualene; hydrocarbon oils of vegetable origin, such as liquid triglycerides of fatty acids having from 6 to 30 carbon atoms such as triglycerides of heptanoic or octanoic acids, or for example sunflower oil, maize oil, soya oil, cucurbit oil, grapeseed oil, sesame oil, hazelnut oil, apricot oil, macadamia oil, arara oil, sunflower oil, castor oil, avocado oil, triglycerides of caprylic/capric acids such as those sold by the company Stearineries Dubois or those sold under the names MIGLYOL® 810, 812 and 818 by the company Dynamit Nobel, jojoba oil, shea butter oil; hydrocarbons with more than 16 carbon atoms, linear or branched, of mineral or synthetic origin, such as paraffin oils, petroleum jelly, liquid paraffin, polydecenes, hydrogenated polyisobutene such as Parleam®. fluorinated, partially hydrocarbon oils; as fluorinated oils, non-limiting examples include perfluoromethylcyclopentane and perfluoro-1,3-dimethylcyclohexane, sold under the names “FLUTEC® PC1” and “FLUTEC® PC3” by the company BNFL Fluorochemicals; perfluoro-1,2-dimethylcyclobutane; perfluoroalkanes such as dodecafluoropentane and tetradecafluorohexane, sold under the names “PF 5050®” and “PF 5060®” by the 3M Company, or bromoperfluorooctyl sold under the name “FORALKYL®” by the company Atochem; nonafluoro-methoxybutane and nonafluoroethoxyisobutane; derivatives of perfluoromorpholine, such as 4-trifluoromethyl perfluoromorpholine sold under the name “PF 5052®” by the 3M Company.

The fatty alcohols usable as fatty substances in the composition of the disclosure include, but are not limited to, non-alkoxylated, saturated or unsaturated, linear or branched, and have from 6 to 30 carbon atoms and more particularly from to 30 carbon atoms; For example, cetyl alcohol, stearyl alcohol and their mixture (cetylstearyl alcohol), octyldodecanol, 2-butyloctanol, 2-hexyldecanol, 2-undecylpentadecanol, oleic alcohol or linoleic alcohol.

The exemplary non-silicone wax or waxes that can be used in the composition of the disclosure are chosen from carnauba wax, candelilla wax, and Alfa wax, paraffin wax, ozokerite, vegetable waxes such as olive wax, rice wax, hydrogenated jojoba wax or absolute waxes of flowers such as the essential wax of blackcurrant flower sold by the company BERTIN (France), animal waxes such as beeswaxes, or modified beeswaxes (cerabellina); other waxes or waxy raw materials usable according to the disclosure are, for example, marine waxes such as that sold by the company SOPHIM under reference M82, waxes of polyethylene or of polyolefins in general.

The exemplary fatty acid esters are the esters of saturated or unsaturated, linear or branched C₁-C₂₆ aliphatic mono- or polyacids and of saturated or unsaturated, linear or branched C₁-C₂₆ aliphatic mono- or polyalcohols, the total number of carbons of the esters being, for example, greater than or equal to 10.

Among the monoesters, non-limiting mentions can be made of dihydroabietyl behenate; octyldodecyl behenate; isocetyl behenate; cetyl lactate; C₁₂-C₁₅ alkyl lactate; isostearyl lactate; lauryl lactate; linoleyl lactate; oleyl lactate; (iso)stearyl octanoate; isocetyl octanoate; octyl octanoate; cetyl octanoate; decyl oleate; isocetyl isostearate; isocetyl laurate; isocetyl stearate; isodecyl octanoate; isodecyl oleate; isononyl isononanoate; isostearyl palmitate; methyl acetyl ricinoleate; myristyl stearate; octyl isononanoate; 2-ethylhexyl isononate; octyl palmitate; octyl pelargonate; octyl stearate; octyldodecyl erucate; oleyl erucate; ethyl and isopropyl palmitates, ethyl-2-hexyl palmitate, 2-octyldecyl palmitate, alkyl myristates such as isopropyl, butyl, cetyl, 2-octyldodecyl, mirystyl, stearyl myristate, hexyl stearate, butyl stearate, isobutyl stearate; dioctyl malate, hexyl laurate, and 2-hexyldecyl laurate.

Further non-limiting mentions of esters can be made of the esters of C₄-C₂₂ di- or tricarboxylic acids and of C₁-C₂₂ alcohols and the esters of mono-, di- or tricarboxylic acids and of C₂-C₂₆ di-, tri-, tetra- or pentahydroxy alcohols.

Even further non-limiting examples of esters include: diethyl sebacate; diisopropyl sebacate; diisopropyl adipate; di-n-propyl adipate; dioctyl adipate; diisostearyl adipate; dioctyl maleate; glyceryl undecylenate; octyldodecyl stearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate; propylene glycol dicaprate, tridecyl erucate; triisopropyl citrate; triisotearyl citrate; glyceryl trilactate; glyceryl trioctanoate; trioctyldodecyl citrate; trioleyl citrate, propylene glycol dioctanoate; neopentyl glycol diheptanoate; diethylene glycol diisanonate; and polyethylene glycol distearates.

Among the esters mentioned above, exemplary esters include ethyl, isopropyl, myristyl, cetyl, stearyl palmitates, ethyl-2-hexyl palmitate, 2-octyldecyl palmitate, alkyl myristates such as isopropyl, butyl, cetyl, 2-octyldodecyl myristate, hexyl stearate, butyl stearate, isobutyl stearate; dioctyl malate, hexyl laurate, 2-hexyldecyl laurate and isononyl isononanate, cetyl octanoate.

The composition can also comprise, as fatty ester, esters and di-esters of sugars of C₆-C₃₀, such as C₁₂-C₂₂ fatty acids. “Sugar” as used in the disclosure means oxygen-containing hydrocarbon compounds that possess several alcohol functions, with or without aldehyde or ketone functions, and having at least 4 carbon atoms. These sugars can be monosaccharides, oligosaccharides or polysaccharides.

As suitable sugars, non-limiting examples include sucrose, glucose, galactose, ribose, fucose, maltose, fructose, mannose, arabinose, xylose, lactose, and their derivatives, for example alkylated, such as methylated derivatives such as methylglucose.

The esters of sugars and of fatty acids can, for example, be chosen from the esters or mixtures of esters of sugars described previously and of linear or branched, saturated or unsaturated C₆-C₃₀, such as C₁₂-C₂₂ fatty acids. If they are unsaturated, these compounds can have one to three, conjugated or unconjugated, carbon-carbon double bonds.

The esters according to at least one embodiment can also be chosen from mono-, di-, tri- and tetra-esters, polyesters and mixtures thereof.

These esters can be for example oleate, laurate, palmitate, myristate, behenate, cocoate, stearate, linoleate, linolenate, caprate, arachidonates, or mixtures thereof such as the oleo-palmitate, oleo-stearate, palmito-stearate mixed esters.

For example, the mono- and di-esters can be used, and such as the mono- or di-oleate, stearate, behenate, oleopalmitate, linoleate, linolenate, oleostearate, of sucrose, of glucose or of methylglucose.

Non-limiting mention can be made of the product sold under the name GLUCATE® DO by the company Amerchol, which is a dioleate of methylglucose.

Exemplary esters or of mixtures of esters of sugar of fatty acid include: the products sold under the names F160, F140, F110, F90, F70, SL40 by the company Crodesta, denoting respectively the palmito-stearates of sucrose formed from 73% of monoester and 27% of di- and tri-ester, from 61% of monoester and 39% of di-, tri-, and tetra-ester, from 52% of monoester and 48% of di-, tri-, and tetra-ester, from 45% of monoester and 55% of di-, tri-, and tetra-ester, from 39% of monoester and 61% of di-, tri-, and tetra-ester, and the mono-laurate of sucrose; the products sold under the name Ryoto Sugar Esters for example with the reference B370 and corresponding to the behenate of sucrose formed from 20% of monoester and 80% of di-triester-polyester; sucrose mono-di-palmito-stearate marketed by the company Goldschmidt under the name TEGOSOFT® PSE.

The silicones usable in the composition of the present disclosure include but are not limited to volatile or non-volatile, cyclic, linear or branched silicones, modified or not with organic groups.

The silicones usable according to the disclosure can be in the form of oils, waxes, resins or gums.

In some embodiments, the silicone is chosen from the polydialkylsiloxanes, such as the polydimethylsiloxanes (PDMS), and the organo-modified polysiloxanes having at least one functional group selected from the poly(alkoxylated) groups, the amine groups and the alkoxy groups.

The organopolysiloxanes are defined in more detail in the work of Walter NOLL “Chemistry and Technology of Silicones” (1968), Academic Press. They can be volatile or non-volatile.

When they are volatile, the silicones are, for example, chosen from those with a boiling point between 60° C. and 260° C., and for further examples, chosen from:

the cyclic polydialkylsiloxanes having from 3 to 7, such as from 4 to 5 silicon atoms. It can be, for example, the octamethylcyclotetrasiloxane marketed under the name VOLATILE SILICONE® 7207 by UNION CARBIDE or SILBIONE® 70045 V2 by RHODIA, the decamethylcyclopentasiloxane marketed under the name VOLATILE SILICONE® 7158 by UNION CARBIDE, and SILBIONE®70045 V5 by RHODIA, and mixtures thereof.

Non-limiting mentions can also be made of the cyclocopolymers of the dimethylsiloxanes/methylalkylsiloxane type, such as SILICONE VOLATILE® FZ 3109 marketed by the company UNION CARBIDE, of the formula I:

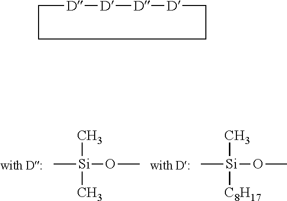

Non-limiting mentions can further be made of the mixtures of cyclic polydialkylsiloxanes with organic compounds derived from silicon, such as the mixture of octamethylcyclotetrasiloxane and tetratrimethylsilylpentaerythritol (50/50) and the mixture of octamethylcyclotetrasiloxane and oxy-1,1′-(hexa-2,2,2′,2′,3,3′-trimethylsilyloxy) bis-neopentane.

Other suitable volatile silicones include the linear volatile polydialkylsiloxanes having 2 to 9 silicon atoms and with a viscosity less than or equal to 5×10⁻⁶ m²/s at 25° C. An example is decamethyltetrasiloxane, marketed under the name “SH 200” by the company TORAY SILICONE. Silicones included in this class are also described in the article published in Cosmetics and Toiletries, Vol. 91, January 76, p. 27-32—TODD BYERS “Volatile Silicone fluids for cosmetics”.

Even further non-limiting mentions can be made of non-volatile polydialkylsiloxanes, gums and resins of polydialkylsiloxanes, polyorganosiloxanes modified with the aforementioned organofunctional groups, and mixtures thereof.

These silicones are, for example, chosen from the polydialkylsiloxanes, such as the polydimethylsiloxanes with trimethylsilyl end groups. The viscosity of the silicones is measured at 25° C. according to standard ASTM 445 Appendix C.

Among these polydialkylsiloxanes, mention can be made of, non-exhaustively, the following commercial products: the SILBIONE® oils of series 47 and 70 047 or the MIRASIL® oils marketed by RHODIA, for example the oil 70 047 V 500 000; the oils of the MIRASIL® series marketed by the company RHODIA; the oils of the 200 series from the company DOW CORNING such as DC200; the VISCASIL® oils from GENERAL ELECTRIC and certain oils of the SF series (SF 96, SF 18) from GENERAL ELECTRIC.

Non-limiting mention can also be made of the polydimethylsiloxanes with dimethylsilanol end groups known under the name of dimethiconol (CTFA), such as the oils of the 48 series from the company RHODIA.

In this class of polydialkylsiloxanes, non-limiting mentions can be made of the products marketed under the names “ABIL WAX® 9800 and 9801” by the company GOLDSCHMIDT, which are polydialkyl (C₁-C₂₀) siloxanes.

The silicone gums usable according to the disclosure are, for example, polydialkylsiloxanes, such as polydimethylsiloxanes with high number-average molecular weights between 200,000 and 1,000,000 used alone or mixed in a solvent. This solvent can be chosen from the volatile silicones, the polydimethylsiloxane (PDMS) oils, the polyphenylmethylsiloxane (PPMS) oils, the isoparaffins, the polyisobutylenes, methylene chloride, pentane, dodecane, tridecane and mixtures thereof.

Products usable according to the disclosure are, for example, mixtures such as: mixtures formed from a chain end hydroxylated polydimethylsiloxane, or dimethiconol (CTFA) and a cyclic polydimethylsiloxane also called cyclomethicone (CTFA), such as the product Q2 1401 marketed by the company DOW CORNING; mixtures of a polydimethylsiloxane gum and a cyclic silicone such as the product SF 1214 Silicone Fluid from the company GENERAL ELECTRIC, said product being a gum SF corresponding to a dimethicone, having a number-average molecular weight of 500,000, dissolved in the oil SF 1202 Silicone Fluid corresponding to decamethylcyclopentasiloxane; mixtures of two PDMS of different viscosities, for example, of a PDMS gum and a PDMS oil, such as the product SF 1236 from the company GENERAL ELECTRIC. The product SF 1236 is a mixture of a gum SE 30 as defined above having a viscosity of 20 m²/s and an oil SF 96 with a viscosity of 5×10⁻⁶ m²/s. This product, for example, has 15% of gum SE 30 and 85% of oil SF 96.

The organopolysiloxane resins usable according to the disclosure include but are not limited to crosslinked siloxane systems containing the units: R₂SiO_{2/2}, R₃SiO_{1/2}, RSiO_{3/2} and SiO_{4/2}

in which R represents an alkyl having 1 to 16 carbon atoms. For example, R denotes a C₁-C₄ lower alkyl group such as methyl.

Among these resins, non-limiting mention can be made of the product marketed under the name “DOW CORNING 593” or those marketed under the names “SILICONE FLUID SS 4230 and SS 4267” by the company GENERAL ELECTRIC, which are silicones of dimethyl/trimethyl siloxane structure.

Non-limiting mention can also be made of the resins of the trimethylsiloxysilicate type, such as those marketed under the names X22-4914, X21-5034 and X21-5037 by the company SHIN-ETSU.

The organomodified silicones usable according to the disclosure include but are not limited to silicones as defined previously, having in their structure at least one organofunctional group fixed by a hydrocarbon group.

In addition to the silicones described above, the organomodified silicones can be polydiaryl siloxanes, such as polydiphenylsiloxanes, and polyalkyl-arylsiloxanes functionalized by the aforementioned organofunctional groups.

The polyalkarylsiloxanes are, for example, chosen from the polydimethyl/methylphenylsiloxanes, the polydimethyl/diphenylsiloxanes, linear and/or branched, with viscosity ranging from 1×10⁻⁵ to 5×10² m²/s at 25° C.

Among these polyalkarylsiloxanes, non-limiting mentions can be made of the products marketed under the following names: the SILBIONE® oils of series 70 641 from RHODIA; the oils of the series RHODORSIL® 70 633 and 763 from RHODIA; the oil DOW CORNING 556 COSMETIC GRADE FLUID from DOW CORNING; the silicones of the PK series from BAYER such as the product PK20; the silicones of the series PN, PH from BAYER such as the products PN1000 and PH1000; certain oils of the SF series from GENERAL ELECTRIC such as SF 1023, SF 1154, SF 1250, SF 1265.

Among the organomodified silicones, non-limiting mention can be made of the polyorganosiloxanes having: polyoxyethylene and/or polyoxypropylene groups optionally with C₆-C₂₄ alkyl groups such as the products called dimethicone copolyol marketed by the company DOW CORNING under the name DC 1248 or the oils SILWET® L 722, L 7500, L 77, L 711 from the company UNION CARBIDE and the alkyl (C₁₂)-methicone copolyol marketed by the company DOW CORNING under the name Q2 5200; substituted or unsubstituted amine groups such as the products marketed under the name GP 4 Silicone Fluid and GP 7100 by the company GENESEE or the products marketed under the names Q2 8220 and DOW CORNING 929 or 939 by the company DOW CORNING. The substituted amine groups are, for example, C₁-C₄ aminoalkyl groups; alkoxylated groups, such as the product marketed under the name “SILICONE COPOLYMER F-755” by SWS SILICONES and ABIL WAX® 2428, 2434 and 2440 by the company GOLDSCHMIDT.

For example, the fatty substance is chosen from compounds that are liquid or pasty at room temperature and at atmospheric pressure.

For further example, the fatty substance is a compound that is liquid at a temperature of 25° C. and at atmospheric pressure.

The fatty substance is, for example, chosen from alkanes, fatty alcohols, esters of fatty acid, esters of fatty alcohol, hydrocarbons, silicones, non-silicone oils, and non-silicone waxes. The non-silicone oils may be selected from mineral, vegetable and synthetic oils.

According to at least one embodiment, the fatty substance is chosen from liquid paraffin, polydecenes, liquid esters of fatty acids and of fatty alcohols, and mixtures thereof.

In some embodiments, the fatty substance is chosen from alkanes, hydrocarbons and silicones.

The liquid fatty substances are advantageously chosen from C₆-C₁₆ alkanes, non-silicone oils of plant, mineral or synthetic origin, liquid fatty alcohols, liquid fatty acids and liquid esters of a fatty acid and/or of a fatty alcohol, or mixtures thereof.

Preferably, the liquid fatty substance is chosen from liquid petroleum jelly, C₆-C₁₆ alkanes, polydecenes, liquid esters of a fatty acid and/or of a fatty alcohol, and liquid fatty alcohols, or mixtures thereof.

A preferred liquid fatty substance for use in the present invention is mineral oil which may be commercially available from the supplier Sonneborn under the tradename Kaydol® Heavy White Mineral Oil or from the supplier Exxonmobil Chemical under the tradename Primol™ 352 or from Sonneborn under the tradename Blandol, or from Armedsa under the tradename Aemoil M-302CG or from Exxonmobil Chemical under the tradename Marcol 82.

In certain embodiments, the at least one fatty substance has a viscosity of about 50 mm²/s or less at 40° C. (kinematic viscosity as measured by the ASTM D 445 method in units of mm²/s at 40° C.)

In other embodiments, the at least one fatty substance has a viscosity of greater than about 50 mm2/s at 40° C. and may be chosen from oils such as mineral oil (kinematic viscosity as measured by the ASTM D 445 method in units of mm2/s at 40° C.)

The at least one fatty substance of the present invention may be employed in an amount of at least about 10% by weight relative to the total weight of the cosmetic composition. For example, the amount of the at least one fatty substance may be from about 10% to about 80% by weight, preferably from about 15% to about 70% by weight, or more preferably from about 20% to about 60% by weight, based on the total weight of the cosmetic composition.

In some embodiments, the at least one fatty substance may be present in an amount of from about 10% to about 60% by weight, preferably from about 20% to about 60% by weight, such as from about 20% to about 40% by weight, or such as from about 20% to about 30% by weight, based on the total weight of the cosmetic composition.

In certain embodiments, the at least one fatty substance may be present in an amount of from about 30% to about 60% by weight, such as at about 30% by weight, or at about 35% by weight, or at about 40%, or at about 45% by weight, or at about 50% by weight, or at about 55% by weight, or at about 60% by weight, based on the total weight of the cosmetic composition.

In some embodiments, when the amount of fatty substances is at about 60% or more by weight, based on the total weight of the cosmetic composition, then the cosmetic composition is in the form of a cream.

In other embodiments, when the amount of fatty substances is from about 40% to less than about 50% by weight, based on the total weight of the cosmetic composition, then the cosmetic composition is in the form of a liquid-cream.

In yet other embodiments, when the amount of fatty substances is less than about 40% by weight, based on the total weight of the cosmetic composition, then the cosmetic composition is in the form of a liquid.

### Acrylic Polymer

The at least one acrylic polymer of the present invention is selected from crosslinked copolymers of (meth)acrylic acid and/or (C1-C6)alkyl esters and from acrylic associative polymers.

The expression “acrylic polymer” is understood, for the purposes of the present invention, to mean a polymer that results from the polymerization of one or more monomers.

The acrylic polymer of the present invention may also belong to a group of compounds known as acrylic thickening polymers.

The expression “thickening polymer” is understood, for the purposes of the present invention, to mean a polymer having, in solution or in dispersion containing 1 percent by weight of active material in water or in ethanol at 25° C., a viscosity greater than 0.2 poise at a shear rate of 1 s-1. The viscosity can be measured with a HAAKE RS600 viscometer from THERMO ELECTRON. This viscometer is a controlled-stress viscometer with cone-plate geometry (for example having a diameter of 60 mm).

As used herein, the term “(meth)acrylic” acid and “(meth)acrylate” are meant to include the corresponding methyl derivatives of acrylic acid and the corresponding alkyl acrylate. For example, “(meth)acrylic)” acid refers to acrylic acid and/or methacrylic acid and “(meth)acrylate” refers to alkyl acrylate and/or alkyl methacrylate.

In certain embodiments, the acrylic polymer of the present invention is selected from crosslinked copolymers of methacrylic acid and of a C1-C6 alkyl ester wherein the C1-C6 alkyl ester is a C1-C6 alkyl acrylate.

Methacrylic acid is preferably present in amounts ranging from 20 percent to 80 percent by weight, more particularly from 25 percent to 70 percent by weight and even more particularly from 35 percent to 65 percent by weight relative to the total weight of the copolymer.

The alkyl acrylate is preferably present in amounts ranging from 15 percent to 80 percent by weight, more particularly from 25 percent to 75 percent by weight and even more particularly from 35 percent to 65 percent by weight relative to the total weight of the copolymer. It is chosen especially from methyl acrylate, ethyl acrylate and butyl acrylate and more particularly ethyl acrylate.

This copolymer is preferably partially or totally/substantially crosslinked with at least one standard polyethylenically unsaturated crosslinking agent, for instance polyalkenyl ethers of sucrose or of polyols, diallyl phthalates, divinylbenzene, allyl (meth)acrylate, ethylene glycol di(meth)acrylate, methylenebisacrylamide, trimethylolpropane tri(meth)acrylate, diallyl itaconate, diallyl fumarate, diallyl maleate, zinc (meth)acrylate, and castor oil or polyol derivatives manufactured from unsaturated carboxylic acids. The content of crosslinking agent generally ranges from 0.01 percent to 5 percent by weight, preferably from 0.03 percent to 3 percent by weight and even more particularly from 0.05 percent to 1 percent by weight relative to the total weight of the copolymer.

In preferred embodiments, the crosslinked copolymer of methacrylic acid and of a C1-C6 alkyl acrylate is slightly cross-linked.

As used herein, the term “slightly crosslinked” refers to a partially crosslinked three-dimensional polymeric network.

In other preferred embodiments, the crosslinked copolymer of methacrylic acid and of a C1-C6 alkyl acrylate is alkali-swellable.

As used herein, the term “alkali-swellable” as it pertains to the acrylic polymer of the present invention refers to a polymer that when introduced to a solution, imparts little or no viscosity, but upon adjusting the pH to mildly acidic, neutral, or mildly basic conditions, a measurable increase in viscosity is observed, i.e., adding an alkali or neutralizing agent to a solution containing an alkali swellable polymer results in the development of viscosity.

The term “alkali-swellable” as used herein may also refer to the expansion of the polymer molecules upon neutralization as a result of charge repulsion of the anionic carboxylate groups of the polymer.

According to one particularly preferred form, the crosslinked copolymer of the invention may especially be in the form of a dispersion of particles in water.

A preferred acrylic polymer of the present invention is selected from a crosslinked (meth)acrylic acid/ethyl acrylate copolymer, a cross-linked anionic acrylate polymer, and mixtures thereof.

According to one particularly preferred form, the acrylic polymer of the present invention selected from a crosslinked (meth)acrylic acid/ethyl acrylate copolymer and a cross-linked anionic acrylate polymer copolymer may especially be in the form of a dispersion in water. The mean size of the copolymer particles in the dispersion is generally between 10 and 500 nm, preferably between 20 and 200 nm and more preferentially from 50 to 150 nm.

In preferred embodiments, the crosslinked (meth)acrylic acid/ethyl acrylate copolymer is a crosslinked methacrylic acid/ethyl acrylate copolymer, also known as an acrylates copolymer in aqueous dispersion, an example of which is a slightly cross-linked, alkali-swellable acrylate polymer known by the INCI name acrylates copolymer and commercially available from the supplier Lubrizol, under the tradename Carbopol® Aqua SF-1 as an aqueous dispersion comprising about 30% by weight of total solids or active material. Carbopol® Aqua SF-1 has a carboxyl functionality in its protonated form. This copolymer belongs to a class of synthetic rheology modifiers that include carboxyl functional alkali-swellable and alkali-soluble thickeners (ASTs). These thickener polymers are prepared from the free-radical polymerization of acrylic acid alone or in combination with other ethylenically unsaturated monomers. The polymers can be synthesized by solvent/precipitation as well as emulsion polymerization techniques.

Other suitable crosslinked (meth)acrylic acid/ethyl acrylate copolymers may be chosen from a crosslinked copolymer of methacrylic acid and of ethyl acrylate as an aqueous dispersion containing 38 percent active material, commercially available from the company Coatex under the name Viscoatex™ 538C or a crosslinked copolymer of acrylic acid and of ethyl acrylate as an aqueous dispersion containing 28 percent active material, commercially available from the company Rohm and Haas and sold under the name Aculyn™ 33.

In other preferred embodiments, the acrylic polymer of the present invention is a cross-linked anionic acrylate polymer. The cross-linked anionic acrylate polymer may be contained in an aqueous dispersion comprising about 32% by weight of total solids. Examples of the cross-linked anionic acrylate polymer of the present invention include, but are not limited to, the polymer known by the INCI name acrylates crosspolymer-4 and commercially available from the supplier Lubrizol, under the tradename Carbopol® Aqua SF-2, as an aqueous dispersion comprising about 32% by weight of total solids or active material. Acrylates Crosspolymer-4 may also be described as a copolymer of acrylic acid, methacrylic acid or one of its simple esters, crosslinked with trimethylolpropane triacrylate.

In certain other embodiments, the acrylic polymer of the present invention is selected from acrylic associative polymers, also known as acryic associative thickeners. The expression “associative thickener” is understood according to the invention to mean an amphiphilic thickener comprising both hydrophilic units and hydrophobic units, in particular comprising at least one C8-C30 fatty chain and at least one hydrophilic unit.

Acrylic associative thickeners that may be used according to the invention are acrylic associative polymers selected from: (i) nonionic amphiphilic polymers comprising at least one fatty chain and at least one hydrophilic unit; (ii) anionic amphiphilic polymers comprising at least one hydrophilic unit and at least one fatty-chain unit; (iii) cationic amphiphilic polymers comprising at least one hydrophilic unit and at least one fatty-chain unit; (iv) amphoteric amphiphilic polymers comprising at least one hydrophilic unit and at least one fatty-chain unit; the fatty chains containing from 10 to 30 carbon atoms.

Preferred acrylic associative polymers of the present invention are acrylic anionic amphiphilic polymers which can be selected from those comprising at least one hydrophilic unit of unsaturated olefinic carboxylic acid type, and at least one hydrophobic unit of (C10-C30) alkyl ester of an unsaturated carboxylic acid type. They are preferably selected from those in which the hydrophilic unit of unsaturated olefinic carboxylic acid type corresponds to the monomer of formula (II) below:

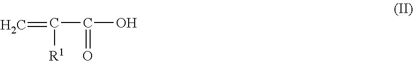

in which formula R1 denotes H or CH3 or C2H5, i.e. acrylic acid, methacrylic acid or ethacrylic acid units, and the hydrophobic unit of which, of (C10-C30)alkyl ester of an unsaturated carboxylic acid type, corresponds to the monomer of formula (III) below:

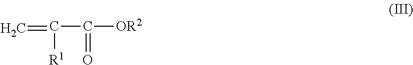

in which formula R1 denotes H or CH3 or C2H5 (i.e. acrylate, methacrylate or ethacrylate units) and preferably H (acrylate units) or CH3 (methacrylate units), R2 denoting a C10-C30 and preferably C12-C22 alkyl radical.

(C10-C30) alkyl esters of unsaturated carboxylic acids according to the invention include, for example, lauryl acrylate, stearyl acrylate, decyl acrylate, isodecyl acrylate and dodecyl acrylate, and the corresponding methacrylates, lauryl methacrylate, stearyl methacrylate, decyl methacrylate, isodecyl methacrylate and dodecyl methacrylate.

Anionic amphiphilic polymers of this type are disclosed and prepared, for example, according to the U.S. Pat. No. 3,915,921 and U.S. Pat. No. 4,509,949.

The anionic amphiphilic polymers that can be used in the context of the present invention may more particularly denote polymers formed from a mixture of monomers comprising:

(i) acrylic acid and one or more esters of formula (IV) below:

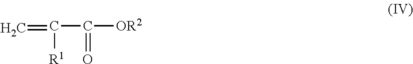

in which R1 denotes H or CH3, R2 denoting an alkyl radical having from 12 to 22 carbon atoms, and a crosslinking agent, such as, for example, those constituted of from 95 percent to 60 percent by weight of acrylic acid (hydrophilic unit), 4 percent to 40 percent by weight of C10-C30 alkyl acrylate (hydrophobic unit), and 0 to 6 percent by weight of crosslinking polymerizable monomer, or 98 percent to 96 percent by weight of acrylic acid (hydrophilic unit), 1 percent to 4 percent by weight of C10-C30 alkyl acrylate (hydrophobic unit) and 0.1 percent to 0.6 percent by weight of crosslinking polymerizable monomer,

(ii) essentially acrylic acid and lauryl methacrylate, such as the product formed from 66 percent by weight of acrylic acid and 34 percent by weight of lauryl methacrylate.

Said crosslinking agent is a monomer containing a

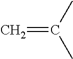

group with at least one other polymerizable group whose unsaturated bonds are not conjugated relative to one another. Mention may be made in particular of polyallyl ethers such as, in particular, polyallyl sucrose and polyallyl pentaerythritol.

Among said polymers above, the ones most particularly preferred according to the present invention are the products sold by the company Goodrich under the trade names Pemulen™ TR1, Pemulen™ TR2, Carbopol® 1382, and more preferably still Pemulen™ TR1, and the product sold by the company Coatex under the name Coatex SX®.

Thus, in some embodiments, the acrylic polymer of the present invention is selected from an acrylate polymer contained in an aqueous dispersion comprising about 30% by weight of active material. This acrylate polymer may be slightly cross-linked and alkali-swellable.

In other embodiments, the acrylic polymer of the present invention is selected from a cross-linked anionic acrylate polymer contained in an aqueous dispersion comprising about 32% by weight of active material.

In yet other embodiments, the acrylic polymer of the present invention is chosen from a slightly cross-linked, alkali-swellable acrylate polymer contained in an aqueous dispersion comprising about 30% by weight of active material, a cross-linked anionic acrylate polymer contained in an aqueous dispersion from comprising about 32% by weight of active material, and mixtures thereof.

In some other embodiments, the acrylic polymer of the present invention is chosen from acrylic associative polymers, in particular, acrylic anionic amphiphilic polymers which can be selected from those comprising at least one hydrophilic unit of unsaturated olefinic carboxylic acid type, and at least one hydrophobic unit of (C10-C30) alkyl ester of an unsaturated carboxylic acid type.

The at least one acrylic polymer of the present invention may be employed in an amount of from about 0.1% to about 10% by weight, preferably from about 0.3% to about 6% by weight, more preferably from about 0.5% to about 5% by weight, even more preferably from about 1% to about 3% by weight, with all weights of the polymer referring to the weight of the active material and based on the total weight of the cosmetic composition of the present invention.

In certain preferred embodiments, the at least one acrylic polymer of the present invention may be employed in an amount of from about 0.2% to about 3% by weight, preferably from about 0.4% to about 2.75% by weight, and more preferably from about 1% to about 2% by weight, preferably, such as at about 2.55%, more preferably, such as at about 1.95% by weight, even more preferably, such as at about 1.65% by weight, or such as at about 1.2% by weight, with all weights of the polymer referring to the weight of the active material and based on the total weight of the cosmetic composition of the present invention.

### Salt

The at least one salt of the present invention may be selected from an ammonium salt other than an ammonium acetate salt, a quaternary ammonium salt, a quaternary diammonium salt, an alkaline earth metal salt, a transition metal salt, an alkali metal salt other than an alkali metal phosphate salt, an agmatine salt, and mixtures thereof.

In some embodiments, the at least one salt of the present invention may be chosen from an alkaline earth metal salt wherein the alkaline earth metal is, for example, beryllium, magnesium, calcium, strontium, or barium.

In other embodiments, the at least one salt of the present invention may be chosen from a transition metal salt wherein the transition metal is, for example, titanium, manganese, zinc, zirconium, hafnium, or aluminum.

In yet other embodiments, the at least one salt of the present invention may be chosen from an alkaline metal salt wherein the alkaline metal is for example, potassium or sodium.

Within the meaning of the present disclosure, “salt” is understood to include, but not limited to, the salts of halides or salts of a mineral acid, such as chlorides, bromides, fluorides and iodides. Also included within this meaning are non-halogenated salts or salts of an organic acid, for example, carboxylic acid salts such as acetates, propionates, pyrrolidonecarboxylates (or pidolates), or sorbates; polyhydroxlated carboxylic acid salts, such as gluconates, heptagluconates, ketogluconates, lactate gluconates, ascorbates or pantothenates; mono- or polycarboxyl hydroxy acid salts, such as citrates or lactates; amino acid salts such as aspartates or glutamates; fulvate salts; hydrogen carbonates (or bicarbonates; carbonates; sulfate salts; hydrogen sulfate salts; nitrates; phosphates; polyphosphates; glycinates; perchlorates; silicates; borates; and salts of carboxylic acids and polymeric complexes which can support said salts, and also their mixtures. It is understood that this term does not cover, within the meaning of the present invention, peroxygenated salts or persalts such as ammonium persulfate or potassium persulfate.

The meaning of “salt” within the meaning of the present disclosure is also understood to include the salts proper that can result from the action of an acid on a metal. The meaning of “salt” within the meaning of the present disclosure is also understood not to include oxides and hydroxides of metals.

In certain embodiments, the at least one salt of the present invention may be chosen from ammonium salts of halides.

In other embodiments, the at least one salt of the present invention may be chosen from ammonium salts of the non-halogenated type other than of the acetate type, such as ammonium acetate salt.

As examples of ammonium salts as used according to the invention, mention may be made especially of ammonium carbonate, ammonium carbamate, ammonium hydrogen carbonate, ammonium sulfates, ammonium glycerophosphates, ammonium chlorides, ammonium nitrates, and mixtures thereof.

Preferably, the ammonium salt(s) are chosen from ammonium chloride and ammonium sulfate, and mixtures thereof. Even more preferentially, the ammonium salt is ammonium chloride.

In other certain embodiments, the at least one salt of the present invention may be chosen from an alkaline earth metal salt, transition metal salt, and an alkaline metal salt other than an alkali metal phosphate salt.

Preferably, the alkaline earth metal salt is a calcium salt. Even more preferentially, the calcium salt is calcium chloride.

Preferably, the transition metal salt is a manganese salt, for example, manganese gluconate.

In preferred embodiments, the alkali metal salt is a sodium or potassium salt of an organic acid or non-halogenated type other than a phosphate salt. Preferably, the alkali metal salt is sodium sulfate or sodium acetate. In preferred embodiments, the alkali metal salt is sodium acetate.

In other embodiments, the at least one salt of the present invention may be chosen from a quaternary ammonium salt and a quaternary diammonium salt.

Suitable examples of quaternary ammonium salts are tetraalkylammonium chlorides, for instance dialkyldimethylammonium or alkyltrimethylammonium chlorides in which the alkyl group contains approximately from 12 to 22 carbon atoms, in particular behenyltrimethylammonium chloride, distearyldimethylammonium chloride, cetyltrimethylammonium chloride, benzyldimethylstearylammonium chloride, or else, secondly, distearoylethylhydroxyethylmethylammonium methosulfate, dipalmitoylethylhydroxyethyl-ammonium methosulfate or distearoylethylhydroxyethylammonium methosulfate, or else, lastly, palmitylamidopropyltrimethylammonium chloride or stearamidopropyl-dimethyl(myristyl acetate) ammonium chloride, sold under the name Ceraphyl® 70 by the company Van Dyk.

Other types of quaternary ammonium salts for use according to the invention are quaternary ammonium salts of imidazoline, di- or triquaternary ammonium salts, and quaternary ammonium salts containing one or more ester functions.

In yet other embodiments, the at least one salt of the present invention may be chosen from agmatine salts, such as for example, agmatine sulfate.

The at least one salt of the present invention can be employed in the cosmetic composition of the present invention in an amount ranging from about 0.5% to about 15% by weight, preferably from about 1% to about 10% by weight, more preferably from about 2% to about 10% by weight, or from about 3% to about 8% by weight, even more preferably from about 5% to about 7.5% by weight, based on the total weight of the composition.

### Cosmetically Acceptable Solvent

The cosmetic compositions of the present invention can comprise other compounds constituting the cosmetically acceptable solvent. This cosmetically acceptable solvent comprises water or a mixture of water and at least one cosmetically acceptable organic solvent.

As examples of organic solvents, non-limiting mentions can be made of monoalcohols and polyols such as ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol.

Other suitable examples of organic solvents are ethylene glycol, propylene glycol, butylene glycol, hexylene glycol, propane diol, and glycerin.

The organic solvents for use in the present invention can be volatile or non-volatile compounds.

The cosmetically acceptable solvent may be employed according to the present invention in an amount ranging from about 5% to about 50% by weight, or such as from about 5% to about 30% by weight, such as from about 5% to about 25% by weight, or such as from about 5% to about 20% by weight, based on the total weight of the cosmetic composition of the present invention.

The organic solvent may be employed according to the present invention in an amount ranging from about 0.1% to about 25% by weight, such as from about 1% to about 15% by weight, or such as from about 3% to about 10% by weight, or such as from about 5% to about 10% by weight, based on the total weight of the cosmetic composition of the present invention.

### Colorants

The cosmetic composition of the present invention may further comprise at least one colorant compound chosen from oxidative dye precursors, direct dyes, pigments, and mixtures thereof.

The oxidation dyes are generally chosen from one or more oxidation bases optionally combined with one or more couplers.

By way of example, the oxidation bases are chosen from para-phenylenediamines, bis(phenyl)alkylenediamines, para-aminophenols, ortho-aminophenols and heterocyclic bases, and the addition salts thereof.

Among the para-phenylenediamines that may be mentioned, for example, are para-phenylenediamine, para-toluenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-methoxymethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N-ethyl-N-(β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 4-aminophenylpyrrolidine, 2-thienyl-para-phenylenediamine, 2-β-hydroxyethylamino-5-aminotoluene and 3-hydroxy-1-(4′-aminophenyl)pyrrolidine, and the addition salts thereof with an acid.

Among the para-phenylenediamines mentioned above, para-phenylenediamine, para-toluenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine and 2-β-acetylaminoethyloxy-para-phenylenediamine, and the addition salts thereof with an acid, are particularly preferred.

Among the bis(phenyl)alkylenediamines that may be mentioned, for example, are N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylenediamine and 1,8-bis(2,5-diaminophenoxy)-3,6-dioxaoctane, and the addition salts thereof.

Among the para-aminophenols that may be mentioned, for example, are para-aminophenol, 4-amino-3-methylphenol, 4-amino-3-fluorophenol, 4-amino-3-chlorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol and 4-amino-2-fluorophenol, and the addition salts thereof with an acid.

Among the ortho-aminophenols that may be mentioned, for example, are 2-aminophenol, 2-amino-5-methylphenol, 2-amino-6-methylphenol and 5-acetamido-2-aminophenol, and the addition salts thereof.

Among the heterocyclic bases that may be mentioned, for example, are pyridine derivatives, pyrimidine derivatives and pyrazole derivatives.

Among the pyridine derivatives that may be mentioned are the compounds described, for example, in patents

GB 1 026 978 and GB 1 153 196, for instance 2,5-diaminopyridine, 2-(4-methoxyphenyl)amino-3-aminopyridine and 3,4-diaminopyridine, and the addition salts thereof.

Other pyridine oxidation bases that are useful in the present invention are the 3-aminopyrazolo[1,5-a]pyridine oxidation bases or the addition salts thereof described, for example, in patent application FR 2 801 308. Examples that may be mentioned include pyrazolo[1,5-a]pyrid-3-ylamine, 2-acetylaminopyrazolo[1,5-a]pyrid-3-ylamine, 2-morpholin-4-ylpyrazolo[1,5-a]pyrid-3-ylamine, 3-aminopyrazolo[1,5-a]pyridine-2-carboxylic acid, 2-methoxypyrazolo[1,5-a]pyrid-3-ylamine, (3-aminopyrazolo[1,5-a]pyrid-7-yl)methanol, 2-(3-aminopyrazolo[1,5-a]pyrid-5-yl)ethanol, 2-(3-aminopyrazolo[1,5-a]pyrid-7-yl)ethanol, (3-aminopyrazolo[1,5-a]pyrid-2-yl)methanol, 3,6-diaminopyrazolo[1,5-a]pyridine, 3,4-diaminopyrazolo[1,5-a]pyridine, pyrazolo[1,5-a]pyridine-3,7-diamine, 7-morpholin-4-ylpyrazolo[1,5-a]pyrid-3-ylamine, pyrazolo[1,5-a]pyridine-3,5-diamine, 5-morpholin-4-ylpyrazolo[1,5-a]pyrid-3-ylamine, 2-[(3-aminopyrazolo[1,5-a]pyrid-5-yl)(2-hydroxyethyl)amino]ethanol, 2-[(3-aminopyrazolo[1,5-a]pyrid-7-yl)(2-hydroxyethyl)amino]ethanol, 3-aminopyrazolo[1,5-a]pyridin-5-ol, 3-aminopyrazolo[1,5-a]pyridin-4-ol, 3-aminopyrazolo[1,5-a]pyridin-6-ol, 3-aminopyrazolo[1,5-a]pyridin-7-ol, 2-□-hydroxyethoxy-3-amino-pyrazolo[1,5-a]pyridine; 2-(4-dimethylpyperazinium-1-yl)-3-amino-pyrazolo[1,5-a]pyridine; and the addition salts thereof.

More particularly oxidation bases that are useful in the present invention are selected from 3-aminopyrazolo-[1,5-a]-pyridines and preferably substituted on carbon atom 2 by:
(a) one (di)(C₁-C₆)(alkyl)amino group wherein said alkyl group can be substituted by at least one hydroxy, amino, imidazolium group;
(b) one heterocycloalkyl group containing from 5 to 7 members chain, and from 1 to 3 heteroatoms, potentially cationic, potentially substituted by one or more (C₁-C₆-alkyl, such as di(C₁-C₄)alkylpiperazinium; or
(c) one (C₁-C₆)alkoxy potentially substituted by one or more hydroxy groups such as □-hydroxyalkoxy, and the addition salts thereof.

Among the pyrimidine derivatives that may be mentioned are the compounds described, for example, in the patents DE 2359399; JP 88-169571; JP 05-63124; EP 0770375 or patent application WO 96/15765, such as 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine and their addition salts and their tautomeric forms, when a tautomeric equilibrium exists.

Among the pyrazole derivatives that may be mentioned are the compounds described in the patents DE 3843892, DE 4133957 and patent applications WO 94/08969, WO 94/08970, FR-A-2 733 749 and DE 195 43 988, such as 4,5-diamino-1-methyl-pyrazole, 4,5-diamino-1-(β-hydroxyethyl)pyrazole, 3,4-diamino-pyrazole, 4,5-diamino-1-(4′-chlorobenzyl)pyrazole, 4,5-diamino-1,3-dimethylpyrazole, 4,5-diamino-3-methyl-1-phenyl-pyrazole, 4,5-diamino-1-methyl-3-phenylpyrazole, 4-amino-1,3-dimethyl-5-hydrazinopyrazole, 1-benzyl-4,5-diamino-3-methyl-pyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methyl-pyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole, 3,5-diamino-4-(β-hydroxyethyl)amino-1-methylpyrazole, and the addition salts thereof. 4,5-Diamino-1-(β-methoxyethyl)pyrazole may also be used.

A 4,5-diaminopyrazole will preferably be used, and even more preferentially 4,5-diamino-1-(β-hydroxyethyl)pyrazole and/or a salt thereof.

Pyrazole derivatives that may also be mentioned include diamino-N,N-dihydropyrazolopyrazolones and especially those described in patent application FR-A-2 886 136, such as the following compounds and the addition salts thereof: 2,3-diamino-6,7-dihydro-1H,5H-pyrazolo[1,2-a]pyrazol-1-one, 2-amino-3-ethylamino-6,7-dihydro-1H,5H-pyrazolo[1,2-a]pyrazol-1-one, 2-amino-3-isopropylamino-6,7-dihydro-1H,5H-pyrazolo[1,2-a]pyrazol-1-one, 2-amino-3-(pyrrolidin-1-yl)-6,7-dihydro-1H,5H-pyrazolo[1,2-a]pyrazol-1-one, 4,5-diamino-1,2-dimethyl-1,2-dihydropyrazol-3-one, 4,5-diamino-1,2-diethyl-1,2-dihydropyrazol-3-one, 4,5-diamino-1,2-di-(2-hydroxyethyl)-1,2-dihydropyrazol-3-one, 2-amino-3-(2-hydroxyethyl)amino-6,7-dihydro-1H,5H-pyrazolo[1,2-a]pyrazol-1-one, 2-amino-3-dimethylamino-6,7-dihydro-1H,5H-pyrazolo[1,2-a]pyrazol-1-one, 2,3-diamino-5,6,7,8-tetrahydro-1H,6H-pyridazino[1,2-a]pyrazol-1-one, 4-amino-1,2-diethyl-5-(pyrrolidin-1-yl)-1,2-dihydropyrazol-3-one, 4-amino-5-(3-dimethylaminopyrrolidin-1-yl)-1,2-diethyl-1,2-dihydropyrazol-3-one, 2,3-diamino-6-hydroxy-6,7-dihydro-1H,5H-pyrazolo[1,2-a]pyrazol-1-one. 2,3-Diamino-6,7-dihydro-1H,5H-pyrazolo[1,2-a]pyrazol-1-one and/or a salt thereof will preferably be used.

4,5-Diamino-1-(β-hydroxyethyl)pyrazole and/or 2,3-diamino-6,7-dihydro-1H,5H-pyrazolo[1,2-a]pyrazol-1-one and/or a salt thereof will preferentially be used as heterocyclic bases.

Composition according to the invention may optionally comprise one or more couplers advantageously chosen from those conventionally used in the dyeing or coloring of keratin fibers.

Among these couplers, mention may be made especially of meta-phenylenediamines, meta-aminophenols, meta-diphenols, naphthalene-based couplers and heterocyclic couplers, and also the addition salts thereof.

Mention may be made, for example, of 2-methyl-5-aminophenol, 5-N-(β-hydroxyethyl)amino-2-methylphenol, 3-aminophenol, 5-amino-6-chloro-o-cresol (3-amino-2-chloro-6-methylphenol), 1,3-dihydroxybenzene, 1,3-dihydroxy-2-methyl-benzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diamino-phenoxy)propane, 3-ureidoaniline, 3-ureido-1-dimethylamino-benzene, sesamol, 1-β-hydroxyethylamino-3,4-methylene-dioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N-(β-hydroxyethyl)amino-3,4-methylene-dioxybenzene, 2,6-bis(J-hydroxyethylamino)toluene, 6-hydroxy-indoline, 2,6-dihydroxy-4-methylpyridine, 1-H-3-methylpyrazol-5-one, 1-phenyl-3-methylpyrazol-5-one, 2,6-dimethyl-pyrazolo[1,5-b]-1,2,4-triazole, 2,6-dimethyl[3,2-c]-1,2,4-triazole and 6-methylpyrazolo[1,5-a]benzimidazole, the addition salts thereof with an acid, and mixtures thereof.

In general, the addition salts of the oxidation bases and couplers that may be used in the context of the invention are especially selected from the addition salts with an acid such as the hydrochlorides, hydrobromides, sulfates, citrates, succinates, tartrates, lactates, tosylates, benzenesulfonates, phosphates and acetates.

The oxidation base(s) each advantageously represent from 0.001% to 10% by weight relative to the total weight of the composition, and preferably from 0.005% to 5% by weight relative to the total weight of the compositions of the present invention.

The coupler(s), if they are present, each advantageously represent from 0.001% to 10% by weight relative to the total weight of the composition, and preferably from 0.005% to 5% by weight relative to the total weight of the compositions of the present invention.

Compositions according to the invention may optionally comprise b) one or more synthetic or natural direct dyes, chosen from anionic and nonionic species, preferably cationic or nonionic species, either as sole dyes or in addition to the oxidation dye(s).

Examples of suitable direct dyes that may be mentioned include azo direct dyes; (poly)methine dyes such as cyanins, hemicyanins and styryls; carbonyl dyes; azine dyes; nitro(hetero)aryl dyes; tri(hetero)arylmethane dyes; porphyrin dyes; phthalocyanin dyes, and natural direct dyes, alone or as mixtures.

Preferably direct dyes are cationic direct dyes. Mention may be made of the hydrazono cationic dyes of formulas (Va) and (V′a), the azo cationic dyes (VIa) and (VI′a) and the diazo cationic dyes (VIIa) below:

Het⁺-C(R^{a})═N—N(R^{a})—Ar,An⁻ (Va)

Het⁺-N(R^{a})—N═C(R^{a})—Ar,An⁻ (V′a)

Het⁺-N═N—Ar,An⁻ (VIa)

Ar⁺—N═N—Ar″,An⁻ (VI′a) and

Het⁺-N═N—Ar′-N═N—Ar,An⁻ (VIIa)

in which formulas (Va), (V′a), (VIa), (VI′a) and (VIIa):
Het⁺ represents a cationic heteroaryl radical, preferably bearing an endocyclic cationic charge, such as imidazolium, indolium or pyridinium, optionally substituted preferentially with one or more (C₁-C₈) alkyl groups such as methyl;
Ar⁺ representing an aryl radical, such as phenyl or naphthyl, bearing an exocyclic cationic charge, preferentially ammonium, particularly tri(C₁-C₈)alkylammonium such as trimethylammonium;
Ar represents an aryl group, especially phenyl, which is optionally substituted, preferentially with one or more electron-donating groups such as i) optionally substituted (C₁-C₈)alkyl, ii) optionally substituted (C₁-C₈)alkoxy, iii) (di)(C₁-C₈)(alkyl)amino optionally substituted on the alkyl group(s) with a hydroxyl group, iv) aryl(C₁-C₈)alkylamino, v) optionally substituted N—(C₁-C₈)alkyl-N-aryl(C₁-C₈)alkylamino or alternatively Ar represents a julolidine group;
Ar′ is an optionally substituted divalent (hetero)arylene group such as phenylene, particularly para-phenylene, or naphthalene, which are optionally substituted, preferentially with one or more groups (C₁-C₈)alkyl, hydroxyl or (C₁-C₈)alkoxy;
Ar″ is an optionally substituted (hetero)aryl group such as phenyl or pyrazolyl, which are optionally substituted, preferentially with one or more groups (C₁-C₈)alkyl, hydroxyl, (di)(C₁-C₈)(alkyl)amino, (C₁-C₈)alkoxy or phenyl;
R^{a} and R^{b}, which may be identical or different, represent a hydrogen atom or a group (C₁-C₈)alkyl, which is optionally substituted, preferentially with a hydroxyl group;
or alternatively the substituent R^{a} with a substituent of Het⁺ and/or R^{b} with a substituent of Ar and/or R^{a} with R^{b} form, together with the atoms that bear them, a (hetero)cycloalkyl;
particularly, R^{a} and R^{b} represent a hydrogen atom or a group (C₁-C₄)alkyl, which is optionally substituted with a hydroxyl group;
An⁻ represents an anionic counter-ion such as mesylate or halide.
   In particular, mention may be made of the azo and hydrazono cationic dyes bearing an endocyclic cationic charge of formulae (Va), (V′a) and (VIa) as defined previously. More particularly those of formulae (Va), (V′a) and (VIa) derived from the dyes described in patent applications WO 95/15144, WO 95/01772 and EP-714954.

Preferentially, the cationic part is derived from the following derivatives:

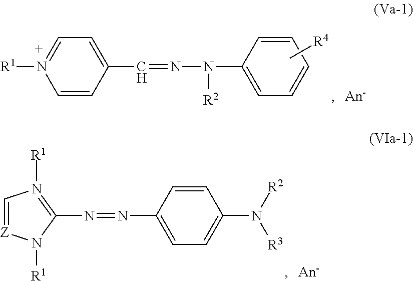

formulae (V-1) and (VI-1) with:
R¹ representing a (C₁-C₄) alkyl group such as methyl;
R² and R³, which are identical or different, represent a hydrogen atom or a (C₁-C₄)alkyl group, such as methyl; and
R⁴ represents a hydrogen atom or an electron-donating group such as optionally substituted (C₁-C₈)alkyl, optionally substituted (C₁-C₈)alkoxy, or (di)(C₁-C₈)(alkyl)amino optionally substituted on the alkyl group(s) with a hydroxyl group; particularly, R⁴ is a hydrogen atom,
Z represents a CH group or a nitrogen atom, preferentially CH;
An⁻ represents an anionic counter-ion such as mesylate or halide.

Particularly, the dye of formulae (Va-1) and (VIa-1) is chosen from Basic Red 51, Basic Yellow 87 and Basic Orange 31 or derivatives thereof:

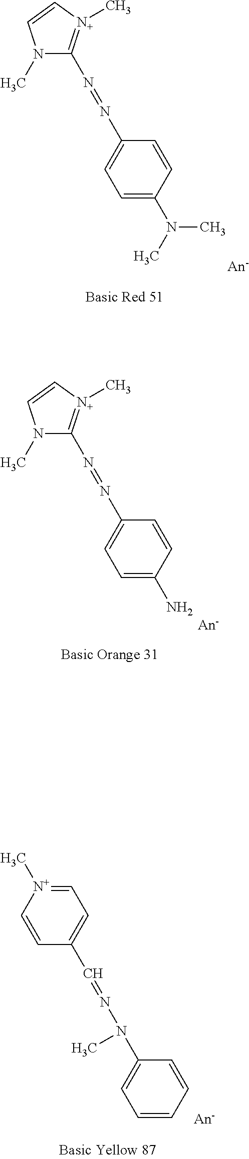

Among the natural direct dyes that may be used according to the invention, mention may be made of lawsone, juglone, alizarin, purpurin, carminic acid, kermesic acid, purpurogallin, protocatechaldehyde, indigo, isatin, curcumin, spinulosin, apigenidin and orceins. Extracts or decoctions containing these natural dyes and in particular henna-based poultices or extracts may also be used.

When they are present, the direct dye(s) more particularly represent from 0.001% to 10% by weight and preferably from 0.005% to 5% by weight of the total weight of the compositions of the present invention.

### Nonionic Surfactants

The cosmetic composition of the present invention can also further comprise at least one nonionic surfactant. In general, nonionic surfactants having a Hydrophilic-Lipophilic Balance (HLB) of from 8 to 20, may be used in the present invention. Non-limiting examples of nonionic surfactants useful in the compositions of the present invention are disclosed in McCutcheon's “Detergents and Emulsifiers,” North American Edition (1986), published by Allured Publishing Corporation; and McCutcheon's “Functional Materials,” North American Edition (1992); both of which are incorporated by reference herein in their entirety.

Examples of nonionic surfactants useful herein include, but are not limited to, alkoxylated derivatives of the following: fatty alcohols, alkyl phenols, fatty acids, fatty acid esters and fatty acid amides, wherein the alkyl chain is in the C₁₂-C₅₀ range, preferably in the C₁₆-C₄₀ range, more preferably in the C₂₄ to C₄₀ range, and having from about 1 to about 110 alkoxy groups. The alkoxy groups are selected from the group consisting of C₂-C₆ oxides and their mixtures, with ethylene oxide, propylene oxide, and their mixtures being the preferred alkoxides. The alkyl chain may be linear, branched, saturated, or unsaturated. Of these alkoxylated non-ionic surfactants, the alkoxylated alcohols are preferred, and the ethoxylated alcohols and propoxylated alcohols are more preferred. The alkoxylated alcohols may be used alone or in mixtures thereof. The alkoxylated alcohols may also be used in mixtures with those alkoxylated materials disclosed herein-above.

Other representative examples of such ethoxylated fatty alcohols include laureth-3 (a lauryl ethoxylate having an average degree of ethoxylation of 3), laureth-23 (a lauryl ethoxylate having an average degree of ethoxylation of 23), ceteth-10 (a cetyl alcohol ethoxylate having an average degree of ethoxylation of 10) steareth-10 (a stearyl alcohol ethoxylate having an average degree of ethoxylation of 10), and steareth-2 (a stearyl alcohol ethoxylate having an average degree of ethoxylation of 2), steareth-100 (a stearyl alcohol ethoxylate having an average degree of ethoxylation of 100), beheneth-5 (a behenyl alcohol ethoxylate having an average degree of ethoxylation of 5), beheneth-10 (a behenyl alcohol ethoxylate having an average degree of ethoxylation of 10), and other derivatives and mixtures of the preceding.

Also available commercially are Brij® nonionic surfactants from Uniqema, Wilmington, Del. Typically, Brij® is the condensation products of aliphatic alcohols with from about 1 to about 54 moles of ethylene oxide, the alkyl chain of the alcohol being typically a linear chain and having from about 8 to about 22 carbon atoms, for example, Brij® 72 (i.e., Steareth-2) and Brij® 76 (i.e., Steareth-10).

Also useful herein as nonionic surfactants are alkyl glycosides, which are the condensation products of long chain alcohols, e.g. C₈-C₃₀ alcohols, with sugar or starch polymers. These compounds can be represented by the formula (S)n-O—R wherein S is a sugar moiety such as glucose, fructose, mannose, galactose, and the like; n is an integer of from about 1 to about 1000, and R is a C₈-C₃₀ alkyl group. Examples of long chain alcohols from which the alkyl group can be derived include decyl alcohol, cetyl alcohol, stearyl alcohol, lauryl alcohol, myristyl alcohol, oleyl alcohol, and the like. Preferred examples of these surfactants are alkyl polyglucosides wherein S is a glucose moiety, R is a C₈-C₂₀ alkyl group, and n is an integer of from about 1 to about 9. Commercially available examples of these surfactants include decyl polyglucoside (available as APG® 325 CS) and lauryl polyglucoside (available as APG® 600CS and 625 CS), all the above-identified polyglucosides APG® are available from Cognis, Ambler, Pa. Also useful herein are sucrose ester surfactants such as sucrose cocoate and sucrose laurate.

Other nonionic surfactants suitable for use in the present invention are glyceryl esters and polyglyceryl esters, including but not limited to, glyceryl monoesters, preferably glyceryl monoesters of C₁₆-C₂₂ saturated, unsaturated and branched chain fatty acids such as glyceryl oleate, glyceryl monostearate, glyceryl monoisostearate, glyceryl monopalmitate, glyceryl monobehenate, and mixtures thereof, and polyglyceryl esters of C₁₆-C₂₂ saturated, unsaturated and branched chain fatty acids, such as polyglyceryl-4 isostearate, polyglyceryl-oleate, polyglyceryl-2 sesquioleate, triglyceryl diisostearate, diglyceryl monooleate, tetraglyceryl monooleate, and mixtures thereof.

Also useful herein as nonionic surfactants are sorbitan esters. Preferable are sorbitan esters of C₁₆-C₂₂ saturated, unsaturated and branched chain fatty acids. Because of the manner in which they are typically manufactured, these sorbitan esters usually comprise mixtures of mono-, di-, tri-, etc. esters. Representative examples of suitable sorbitan esters include sorbitan monooleate (e.g., SPAN® 80), sorbitan sesquioleate (e.g., Arlacel® 83 from Uniqema, Wilmington, Del.), sorbitan monoisostearate (e.g., CRILL® 6 from Croda, Inc., Edison, N.J.), sorbitan stearates (e.g., SPAN® 60), sorbitan trioleate (e.g., SPAN® 85), sorbitan tristearate (e.g., SPAN® 65), sorbitan dipalmitates (e.g., SPAN® 40), and sorbitan isostearate. Sorbitan monoisostearate and sorbitan sesquioleate are particularly preferred emulsifiers for use in the present invention.

Also suitable for use herein are alkoxylated derivatives of glyceryl esters, sorbitan esters, and alkyl polyglycosides, wherein the alkoxy groups is selected from the group consisting of C₂-C₆ oxides and their mixtures, with ethoxylated or propoxylated derivatives of these materials being the preferred. Nonlimiting examples of commercially available ethoxylated materials include TWEEN® (ethoxylated sorbitan mono-, di- and/or tri-esters of C₁₂ to C₁₈ fatty acids with an average degree of ethoxylation of from about 2 to about 20).

In preferred embodiments, the nonionic surfactant(s) for use in the compositions of the present invention is other than the above-described fatty substance(s) employed in said compositions.

Preferred nonionic surfactants are those formed from a fatty alcohol, a fatty acid, or a glyceride with a C₄ to C₃₆ carbon chain, preferably a C₁₂ to C₁₈ carbon chain, more preferably a C₁₆ to C₁₈ carbon chain, derivatized to yield an HLB of at least 8. HLB is understood to mean the balance between the size and strength of the hydrophilic group and the size and strength of the lipophilic group of the surfactant. Such derivatives can be polymers such as ethoxylates, propoxylates, polyglucosides, polyglycerins, polylactates, polyglycolates, polysorbates, and others that would be apparent to one of ordinary skill in the art. Such derivatives may also be mixed polymers of the above, such as ethoxylate/propoxylate species, where the total HLB is preferably greater than or equal to 8. Preferably the nonionic surfactants contain ethoxylate in a molar content of from 10-25, more preferably from 10-20 moles.

The nonionic surfactant will typically be present in the cosmetic composition in an amount of from about 0.1% to about 30% by weight, preferably from about 0.5% to 20% by weight, and more preferably from about 1% to about 10% by weight, such as from about 1% to about 5% by weight, based on the total weight of the composition.

### Neutralizing Agents

In preferred embodiments, the at least one acrylic polymer of the present invention is neutralized in water or in an aqueous solution with a neutralizing agent before the polymer is added into the cosmetic composition of the present invention.

In other preferred embodiments, the at least one acrylic polymer of the present invention is neutralized with a neutralizing agent at the time of addition of the polymer into the cosmetic composition of the present invention.

The neutralizing agent is employed in an amount sufficient to neutralize the acrylic polymer of the present invention in water or an aqueous solution. After neutralization, the acrylic polymer may be partially or fully neutralized. One indication of neutralization is the clarity of the solution.

Suitable neutralizing agents are other than the at least one salt described above and may be selected from alkali metal carbonates, alkali metal phosphates, organic amines, hydroxide base compounds, and mixtures thereof, particularly from ethylamines, ethyleneamines, alkanolamines, cyclic amines and other cyclic compounds, saturated or unsaturated, having one or more nitrogen atoms within the ring.

The organic amines may be chosen from the ones having a pKb at 25° C. of less than 12, such as less than 10 or such as less than 6. It should be noted that this is the pKb corresponding to the function of highest basicity.

Organic amines may be chosen from organic amines comprising one or two primary, secondary, or tertiary amine functions, and at least one linear or branched C₁-C₈ alkyl groups bearing at least one hydroxyl radical.

Organic amines may also be chosen from alkanolamines such as mono-, di- or trialkanolamines, comprising one to three identical or different C₁-C₄ hydroxyalkyl radicals, ethylamines, ethyleneamines, quinoline, aniline and cyclic amines, such as pyrroline, pyrrole, pyrrolidine, imidazole, imidazolidine, imidazolidinine, morpholine, pyridine, piperidine, pyrimidine, piperazine, triazine and derivatives thereof.

Among the compounds of the alkanolamine type that may be mentioned include but not limited to: monoethanolamine (also known as monoethanolamine or MEA), diethanolamine, triethanolamine, monoisopropanolamine, diisopropanolamine, N-dimethylaminoethanolamine, 2-amino-2-methyl-1-propanol, triisopropanolamine, 2-amino-2-methyl-1,3-propanediol, 3-amino-1,2-propanediol, 3-dimethylamino-1,2-propanediol, 2-amino-2-methyl-1-propanol, and tris(hydroxymethylamino)methane.

Other examples include but are not limited to: 1,3-diaminopropane, 1,3-diamino-2-propanol, spermine, and spermidine.

In some embodiments, the organic amines are chosen from amino acids.

As non-limiting examples, the amino acids that may be used may be of natural or synthetic origin, in L, D, or racemic form, and comprise at least one acid function chosen from, for instance, carboxylic acid, sulfonic acid, phosphonic acid, and phosphoric acid functions. The amino acids may be in their neutral or ionic form.

Amino acids that may be used in the present disclosure include but are not limited to: aspartic acid, glutamic acid, alanine, arginine, ornithine, citrulline, asparagine, carnitine, cysteine, glutamine, glycine, histidine, lysine, isoleucine, leucine, methionine, N-phenylalanine, proline, serine, taurine, threonine, tryptophan, tyrosine, and valine.

Further as non-limiting examples, the amino acids may be chosen from basic amino acids comprising an additional amine function optionally included in a ring or in a ureido function. Such basic amino acids may be chosen from histidine, lysine, arginine, ornithine, and citrulline.

In some embodiments, the organic amines are chosen from organic amines of heterocyclic type. Besides histidine that has already been mentioned in the amino acids, non-limiting mention may also be made of pyridine, piperidine, imidazole, 1,2,4-triazole, tetrazole, and benzimidazole.

In some embodiments, the organic amines are chosen from amino acid dipeptides. Amino acid dipeptides that may be used in the present disclosure include but not limited to: carnosine, anserine, and baleine.

In some embodiments, the organic amines are chosen from compounds comprising a guanidine function. Organic amines of this type that may be used in the present disclosure include, besides arginine that has already been mentioned as an amino acid, creatine, creatinine, 1,1-dimethylguanidine, 1,1-diethylguanidine, glycocyamine, metformin, agmatine, N-amidinoalanine, 3-guanidinopropionic acid, 4-guanidinobutyric acid, and 2-([amino(imino)methyl]amino)ethane-1-sulfonic acid.

The alkali metal phosphates and carbonates that may be used are, for example, sodium phosphate, potassium phosphate, sodium carbonate, sodium bicarbonate, potassium carbonate, potassium bicarbonate, and their derivatives.

The hydroxide base compounds chosen from alkali metal hydroxides, alkaline-earth metal hydroxides, transition metal hydroxides, quaternary ammonium hydroxides, organic hydroxides, and mixtures thereof. Suitable examples are ammonium hydroxide, sodium hydroxide, potassium hydroxide, lithium hydroxide, rubidium hydroxide, caesium hydroxide, francium hydroxide, beryllium hydroxide, magnesium hydroxide, calcium hydroxide, strontium hydroxide, barium hydroxide, molybdenum hydroxide, manganese hydroxide, zinc hydroxide, cobalt hydroxide, cadmium hydroxide, cerium hydroxide, lanthanum hydroxide, actinium hydroxide, thorium hydroxide, aluminium hydroxide, guanidinium hydroxide and mixtures thereof.

According to at least one embodiment, the neutralizing agent is chosen from at least one organic amine such as at least one alkanolamine. Particularly preferred alkanolamines are ethanolamine (also known as monoethanolamine or MEA), triethanolamine, and 2-amino-2-methyl-1-propanol, and mixtures thereof. An even more particularly preferred alkanolamine is ethanolamine.

The at least one neutralizing agent of the present invention may be employed in an amount of from about 0.01% to about 10% by weight, or such as from about 0.1% to about 5% by weight, or such as from about 0.5% to about 3% by weight, on the total weight of the cosmetic composition of the present invention.

In preferred embodiments, the at least one neutralizing agent of the present invention may be employed in an amount of from about 0.01% to about 1% by weight, or preferably from about 0.5% to about 1% by weight, based on the total weight of the cosmetic composition of the present invention.

### Oxidizing Agent

The present invention requires an oxidizing composition including at least one oxidizing agent which may be chosen, for example, from peroxides, persulfates, perborates percarbonates, alkali metal bromates, ferricyanides, peroxygenated salts, or a mixture thereof. Oxidizing agents that may also be used include at least one redox enzyme such as laccases, peroxidases, and 2-electron oxidoreductases, such as uricase, where appropriate in the presence of their respective donor or co-factor. Oxygen in the air may also be employed as an oxidizing agent.

In one embodiment, the oxidizing agent is hydrogen peroxide present in an aqueous solution whose titre may range from 1 to 40 volumes, such as from 5 to 40 volumes or such as from 5 to 20 volumes.

In another embodiment, the oxidizing agent is a persulfate and/or a monopersulfate such as, for example, potassium persulfate, sodium persulfate, ammonium persulfate, as well as mixtures thereof. In one embodiment, the oxidizing agents in the present disclosure are selected from hydrogen peroxide, potassium persulfate, sodium persulfate, and mixtures thereof.

In particularly preferred embodiments, the oxidizing agent is hydrogen peroxide.

In general, the oxidizing agent will be present in an amount of from about 0.05 to about 50% by weight, such as from about 0.1% to about 30% by weight, or such as from about 0.1% to about 20% by weight, or such as from about 1% to about 10% by weight, based on the total weight of the oxidizing composition.

In one particular embodiment, the oxidizing composition is aqueous or is in the form of an emulsion.

In another embodiment, the oxidizing composition is substantially anhydrous.

The term “substantially anhydrous” means that the oxidizing composition is either completely free of water or contains no appreciable amount of water, for example, no more than 5% by weight, or no more than 2% by weight, or no more than 1% by weight, based on the weight of the oxidizing composition. It should be noted that this refers for example to bound water, such as the water of crystallization of the salts or traces of water absorbed by the raw materials used in the preparation of the compositions according to the disclosure.

The oxidizing composition can contain at least one solvent, chosen from water, organic solvents, and mixtures thereof.

When the oxidizing composition is substantially anhydrous, the oxidizing composition may comprise at least one solvent chosen from organic solvents.

Suitable organic solvents for use in the oxidizing composition include ethanol, isopropyl alcohol, propanol, benzyl alcohol, phenyl ethyl alcohol, glycols and glycol ethers, such as propylene glycol, hexylene glycol, ethylene glycol monomethyl, monoethyl or monobutyl ether, propylene glycol and its ethers, such as propylene glycol monomethyl ether, butylene glycol, dipropylene glycol, diethylene glycol alkyl ethers, such as diethylene glycol monoethyl ether and monobutyl ether, ethylene glycol, propylene glycol, butylene glycol, hexylene glycol, propane diol, glycerin, hydrocarbons such as straight chain hydrocarbons, mineral oil, polybutene, hydrogenated polyisobutene, hydrogenated polydecene, polydecene, squalane, petrolatum, isoparaffins, and mixtures, thereof.

The organic solvents for use in the present invention can be volatile or non-volatile compounds.

The organic solvent may, for example, be present in an amount ranging from about 0.5% to about 70% by weight, such as from about 2% to about 60% by weight, preferably from about 5 to about 50% by weight, relative to the total weight of the oxidizing composition.

The oxidizing composition may be in the form of a powder, gel, liquid, foam, lotion, cream, mousse, and emulsion.

The oxidizing composition of the present invention my also contain at least one fatty substance as described above. Thus, the total amount of fatty substances in the combination or mixture of the cosmetic and oxidizing compositions of the present invention may range from about 10% to about 80% by weight, or such as from about 20% to about 60% by weight, or such as from about 20% to about 40% by weight, or such as from about 20% to about 30% by weight, based on the total weight of the composition.

pH

In some embodiments, the pH of the cosmetic composition is greater than or equal to 7 and may range from about 7.1 to about 12, or such as from about 7.5 to about 11, or such as from about 7.5 to about 10 or such as from about 7.5 to about 9, or such as from about 9 to about 11.

In other embodiments, the pH of the cosmetic composition of the present invention is less than 7 and can range from about 2 to 6.9, or such as from about 2 to about 6.8 or such as from about 3 to about 6.5 or such as from about 3 to about 6 or such as from about 4 to about 6.

The pH of the cosmetic composition may be adjusted to the desired value using the neutralizing agents of the present invention and/or acidifying or basifying agents that are well known in the cosmetic arts.

The pH of the oxidizing composition can range from about 2 to about 12, such as from about 6 to about 11, and it may be adjusted to the desired value using acidifying/alkalizing agents that are well known in the art. In certain embodiments, the pH of the oxidizing composition is below 7.

The pH of the composition resulting from mixing together the cosmetic composition and the oxidizing composition, may range from about 2 to about 7, such as from about 3 to about 6.9, or from about 4 to about 6.9, or from about 4 to about 6.85, or from about 5 to about 6.8.

All numbers expressing pH values are to be understood as being modified in all instances by the term “about” which encompasses up to +3%.

According to at least one embodiment, the cosmetic compositions and compositions comprising the cosmetic composition and the oxidizing composition of the present invention are substantially free of ammonia.

The cosmetic composition of the present disclosure is preferably in the form of an emulsion, for example, oil-in-water emulsion and water-in-oil emulsion.

In particularly preferred embodiments, the cosmetic composition of the present disclosure is preferably in the form of an oil-in-water emulsion.

The cosmetic and oxidizing compositions of the present invention may further comprise at least one auxiliary agent suitable for use in cosmetic compositions. The auxiliary agent may include, but is not limited to thickening agents and rheology modifying polymers other than the acrylic polymers described above, cationic polymers, film forming polymers, humectants and moisturizing agents, emulsifying agents other than those that fall under the above-described fatty substances, fillers, structuring agents, propellants, anionic surfactants, cationic surfactants, amphoteric surfactants, shine agents, and conditioning agents.

Thickening agents and rheology modifying polymers other than the above-described acrylic polymers may be chosen from polymeric thickeners and non-polymeric thickeners. The polymeric thickener can be chosen from ionic or non-ionic, associative or non-associative polymers. Exemplary polymeric thickeners include various native gums. Representative non-polymeric thickening agents include oxyethylenated molecules and especially ethoxylated alkyl or acyl derivatives of polyols. These polymers can be modified physically or chemically.

The at least one thickening agent may be employed in the compositions of the present invention in an amount of from greater than 0% to about 15% by weight, preferably from about 0.1% to about 10% by weight, and more preferably from about 1% to about 5% by weight, based on the total weight of the compositions of the present invention.

The compositions according to the present invention can also comprise at least one cationic polymer.

The cationic polymer may be chosen from cationic associative polymers comprising, in their structure, a pendent or terminal hydrophobic chain, for example of alkyl or alkenyl type, containing from 10 to 30 carbon atoms.

The at least one cationic polymer of the compositions can also be chosen from, for example:

(1) homopolymers and copolymers derived from acrylic or methacrylic esters or amides, examples of which are:

copolymers of acrylamide and of dimethylaminoethyl acrylate quaternized with dimethyl sulfate or with a dimethyl halide, such as the product sold under the name HERCOFLOC by the company Hercules,

the copolymers of acrylamide and of methacryloyloxyethyltrimethylammonium chloride described, for example, in EP 80 976 and sold under the name BINA QUAT P 100 by the company Ciba Geigy,

the copolymer of acrylamide and of methacryloyloxyethyltrimethylammonium methosulfate sold under the name RETEN by the company Hercules,

quaternized or non-quaternized vinylpyrrolidone/dialkylaminoalkyl acrylate or acrylate copolymers, such as the products sold under the name GAFQUAT by the company ISP, for instance GAFQUAT 734 or GAFQUAT 755, or alternatively the products known as COPOLYMER 845, 958 and 937,

dimethylaminoethyl acrylate/vinylcaprolactam/vinylpyrrolidone terpolymers, such as the product sold under the name GAFFIX VC 713 by the company ISP,

vinylpyrrolidone/methacrylamidopropyldimethylamine copolymers sold, for example, under the name STYLEZE CC 10 by ISP,

quaternized vinylpyrrolidone/dimethylaminopropylmethacrylamide copolymers such as the product sold under the name GAFQUAT HS 100 by the company ISP, and crosslinked polymers of methacryloyloxy(C₁-C₄)alkyltri(C₁-C₄)alkylammonium salts such as the polymers obtained by homopolymerization of dimethylaminoethyl acrylate quaternized with methyl chloride, or by copolymerization of acrylamide with dimethylaminoethyl acrylate quaternized with methyl chloride, the homo- or copolymerization being followed by crosslinking with a compound containing olefinic unsaturation, such as methylenebisacrylamide. In at least one embodiment, a crosslinked acrylamide/methacryloyloxyethyltrimethylammonium chloride copolymer (20/80 by weight) in the form of a dispersion containing 50% by weight of the copolymer in mineral oil can be used. This dispersion is sold under the name SALCARE® SC 92 by the company Ciba. In some embodiments, a crosslinked methacryloyloxyethyltrimethylammonium chloride homopolymer containing about 50% by weight of the homopolymer in mineral oil or in a liquid ester can be used. These dispersions are sold under the names SALCARE® SC 95 and SALCARE® SC 96 by the company Ciba.

Other examples are cellulose ether derivatives comprising quaternary ammonium groups, such as the polymers sold under the names JR (JR 400, JR 125, JR 30M) or LR (LR 400, LR 30M) by the company Union Carbide Corporation.

(2) copolymers of cellulose or cellulose derivatives grafted with a water-soluble quaternary ammonium monomer, such as hydroxymethyl-, hydroxyethyl- or hydroxy-propylcelluloses grafted, for instance, with a methacryloylethyltrimethylammonium, methacrylamidopropyltrimethylammonium or dimethyldiallylammonium salt. These are sold under the name CELQUAT L 200 and CELQUAT H 100 by the company National Starch.

(3) non-cellulose cationic polysaccharides, such as guar gums containing trialkylammonium cationic groups. Such products are sold, for example, under the trade names JAGUAR C13S, JAGUAR C15, JAGUAR C17 and JAGUAR C162 by the company Meyhall.

(4) polymers of piperazinyl units and of divalent alkylene or hydroxyalkylene radicals.

(5) water-soluble polyamino amides prepared, for example, by polycondensation of an acidic compound with a polyamine; these polyamino amides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or alternatively with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative; the crosslinking agent being used in an amount ranging from 0.025 to 0.35 mol per amine group of the polyamino amide; these polyamino amides can be alkylated or, if they contain at least one tertiary amine function, they can be quaternized. Exemplary mention may be made of the adipic acid/dimethylaminohydroxypropyl/diethylenetriamine polymers sold under the name CARTARETINE F, F4 or F8 by the company Sandoz.

(6) the polymers obtained by reaction of at least one polyalkylene polyamine containing two primary amine groups and at least one secondary amine group with a dicarboxylic acid chosen from diglycolic acid and saturated C₃-C₈ aliphatic dicarboxylic acids. The molar ratio between the polyalkylene polyamine and the dicarboxylic acid ranges from 0.8:1 to 1.4:1; the polyamino amide resulting therefrom is reacted with epichlorohydrin in a molar ratio of epichlorohydrin relative to the secondary amine group of the polyamino amide ranging from 0.5:1 to 1.8:1. Polymers of this type are sold, for example, under the name HERCOSETT 57, PD 170 or DELSETTE 101 by the company Hercules.

(7) cyclopolymers of alkyldiallylamine and of dialkyldiallylammonium, such as for example: dimethyldiallylammonium chloride homopolymer sold under the name MERQUAT® 100 and MERQUAT® 280 by the company Nalco (and its homologues of low weight-average molecular mass) and the copolymers of diallyldimethylammonium chloride and of acrylamide, sold under the name MERQUAT® 550.

(8) quaternary diammonium polymers.

(9) polyquaternary ammonium polymers; examples that may be mentioned include the products MIRAPOL A 15, MIRAPOL AD1, MIRAPOL AZ1 and MIRAPOL 175 sold by the company Miranol.

(10) quaternary polymers of vinylpyrrolidone and of vinylimidazole, for instance the products sold under the names LUVIQUAT FC 905, FC 550 and FC 370 by the company BASF.

(11) vinylamide homopolymers or copolymers, such as partially hydrolysed vinylamide homopolymers such as poly(vinylamine/vinylamide)s.

(12) cationic polyurethane derivatives, for example those of elastic nature formed from the reaction:

(a1) of at least one cationic unit resulting from at least one tertiary or quaternary amine bearing at least two reactive functions containing labile hydrogen,

(a2) of at least one mixture of at least two different nonionic units bearing at least two reactive functions containing labile hydrogen, for instance chosen from hydroxyl groups, primary or secondary amine groups, and thiol groups, and

(b) of at least one compound comprising at least two isocyanate functions.

(13) Other cationic polymers that may be used in the context of the disclosure include, for example, cationic proteins or cationic protein hydrolysates, polyalkyleneimines, such as polyethyleneimines, polymers containing vinylpyridine or vinylpyridinium units, and chitin derivatives.

Particularly useful cationic polymers in the present invention include, but are not limited to, polyquaternium 4, polyquaternium 6, polyquaternium 7, polyquaternium 10, polyquaternium 11, polyquaternium 16, polyquaternium 22, polyquaternium 28, polyquaternium 32, polyquaternium-46, polyquaternium-51, polyquaternium-52, polyquaternium-53, polyquaternium-54, polyquaternium-55, polyquaternium-56, polyquaternium-57, polyquaternium-58, polyquaternium-59, polyquaternium-60, polyquaternium-63, polyquaternium-64, polyquaternium-65, polyquaternium-66, polyquaternium-67, polyquaternium-70, polyquaternium-73, polyquaternium-74, polyquaternium-75, polyquaternium-76, polyquaternium-77, polyquaternium-78, polyquaternium-79, polyquaternium-80, polyquaternium-81, polyquaternium-82, polyquaternium-84, polyquaternium-85, polyquaternium-86, polyquaternium-87, polyquaternium-90, polyquaternium-91, polyquaternium-92, polyquaternium-94, and guar hydroxypropyltrimonium chloride.

Particularly preferred cationic polymers of the present invention include POLYMER JR-125, POLYMER JR-400, Polymer JR-30M hydroxyethyl cellulosic polymers (polyquaternium 10) available from AMERCHOL; JAGUAR C® 13-S, guar hydroxypropyltrimonium chloride, available from Rhodia; and MERQUAT® 100 and 280, a dimethyl dialkyl ammonium chloride (polyquaternium 6) available from Nalco.

The cationic polymer is generally present in an amount of from greater than 0% to about 15%, preferably from about 0.5 to about 10% by weight, and more preferably from about 1 to about 5% by weight, based on the total weight of the compositions of the present invention.

The compositions of the present invention according to the disclosure can also comprise at least one additive used conventionally in compositions for application onto hair.

“Additive” means a substance that is added, different from the compounds already mentioned.

As examples of additives that can be used, non-limiting mentions can be made of antioxidants or reducing agents, penetrating agents, sequestering agents, perfumes, buffers, dispersants, ceramides, sunscreen agents, preservatives, opacifiers, and antistatic agents.

The cosmetic and oxidizing compositions of the present invention according to the disclosure can be in various forms, such as in the form of liquids, creams, liquid-gels, liquid-creams, gels, lotions or pastes.

It was surprisingly and unexpectedly discovered that the use of nonionic surfactants helped stabilize the cosmetic composition (i.e., there was no phase separation) when oxidative dye precursors are present in the composition. Thus, in preferred embodiments, when colorant compounds such as oxidative dye precursors are present in the cosmetic composition, at least one nonionic surfactant is also present. Preferably, the nonionic surfactant is chosen from alkoxylated nonionic surfactants.

In preferred embodiments, the process of making the cosmetic composition involves a cold process that does not require the use of heat while the ingredients are mixed and does not require the use of an emulsifier blade.

In other preferred embodiments, the cosmetic composition does not require the presence of a fatty alcohol in order to form an emulsion. When the cosmetic composition does not contain a fatty alcohol, the cosmetic composition may be prepared in the absence of heat using mixing speeds at shearing rates that are lower than the shearing rates used to prepare conventional/commercial hair lighteners that are typically provided in the form of emulsions.

According to the present disclosure, the process of making the cosmetic composition involves the general procedure of:

mixing the acrylic polymer (e.g, Carbopol® Aqua SF-1 or Carbopol® Aqua SF-2) with water using a mixer,

if necessary, adding an amount of a neutralizing agent, such as ethanolamine, to the mixture, in order to neutralize the acrylic polymer (as indicated by the formation of a clear solution),

adding a fatty substance (e.g., mineral oil) and optionally, nonionic surfactant (s) while mixing, wherein the speed of mixing can be increased to ensure proper mixing),

optionally, adding more of the fatty substance and/or another fatty substance and/or an emulsifying agent if an emulsion is desired, while continuously mixing,

optionally, adding an organic solvent (e.g. ethanol and/or glycerin and/or propylene glycol),

adding salt (e.g., ammonium chloride) and optionally, dyes or colorant compounds, auxiliary ingredients, additional fatty substances, and antioxidants e.g., sodium bisulfite and erythrobic acid.

The acrylic polymer may also be pre-neutralized before it is combined with the other ingredients according to the process above.

The above-described process is a cold process, that is, it does not require heat and reduces the amount of time needed to prepare a conventional/commercial hair cosmetic composition.

### Process of Lifting the Color of Hair or Altering the Color of Hair

The cosmetic compositions of the present invention are capable of being mixed with an oxidizing composition containing at least oxidizing agent.

The term “mixed” and all variations of this term as used herein refers to contacting or combining or reconstituting or dissolving or dispersing or blending or shaking the cosmetic composition with the oxidizing composition. It can also mean introducing the cosmetic composition to the oxidizing composition. It may also mean placing the cosmetic composition in the same vessel or container as the oxidizing composition.

Thus, the process of lifting or altering the color of hair in accordance with the invention comprises applying a composition comprising the cosmetic composition and the oxidizing composition of the present invention onto hair. Said composition that is applied onto hair is formed by mixing the cosmetic composition with the oxidizing composition.

The cosmetic composition can be mixed or combined with the oxidizing composition in a ratio by weight of from about 1:1 to about 1:10, such as from about 1:1 to about 1:4, preferably from about 1:1 to about 1:3, or preferably from about 1:1 to about 1:2.

Upon application of the composition comprising the cosmetic composition and the oxidizing composition and after a resting time (leave-on time) on the keratin fibers, for example, ranging from about 1 to about 60 minutes, such as from about 5 to about 45 minutes, or such as from about 5 to about 20 minutes, or such as from about 10 to about 20 minutes, or such as of about 20 minutes, the keratin fibers are rinsed, optionally washed with shampoo, rinsed again, optionally washed with a hair conditioning composition, and rinsed again, then dried. The shampoo and hair conditioning composition can be any conventional hair shampoo and conditioner products.

In addition, independently of the embodiment use, the mixture or composition present on the fibers or hair (resulting from the extemporaneous mixing of the compositions, or from the successive application of the cosmetic and oxidizing compositions) is left in place for a time, generally, from about 1 to about 60 minutes, such as from about 5 to about 45 minutes, or such as from about 5 to about 20 minutes, or such as from about 10 to about 20 minutes, or such as of about 20 minutes.

The temperature during the process of lifting or altering the color of hair is between room temperature and 80° C. and preferably, between room temperature and 60° C.

It has been surprisingly discovered that the combination of the fatty substance, the acrylic polymer, the salt and a cosmetically acceptable solvent results in a stable cream or liquid emulsion cosmetic composition which, when combined with the oxidizing composition of the present invention, produces a final mixture or a composition with a non-drip consistency that is still easy to spread on keratin fibers, such as hair.

It has also been discovered that the application of the final mixture or composition onto the fibers results in satisfactory lifting or lightening of the color of the fibers. When the cosmetic composition of the present invention further comprises a colorant compound selected from oxidative dye precursors, direct dyes, pigments or their mixtures, the fibers are also colored satisfactorily with respect to degree of color deposit and desirable shade formation coloring. At the same time, lower amounts of the oxidizing agent and/or lower amounts of oxidative dye precursors and dye compounds can be used compared to conventional dyeing compositions.

The coloring obtained using the compositions and process of the present disclosure may also be durable or wash/fade resistant.

The lifting of the color of the hair is evaluated by the tone height or level which describes the degree or level of lift or lightening. The tone heights range from 1 (black) to 10 (light blond), one unit corresponding to one tone; the higher the number, the lighter the shade or the greater the degree of lift.

It was surprisingly and unexpectedly discovered that by using the compositions and process of the present invention on hair, the color of the hair was sufficiently lightened such that the degree of lift (increase in tone height) ranged from 0.5 to 4, such as from 1 to 3 or such as from 1 to 2. For example, when the starting tone height before treating the hair is 5, and the tone height after treating the hair is 6, then the degree of lift or increase in tone height is 1. At the same time, the hair treated with the compositions of the invention did not feel as rough and did not visually appear to be as damaged as hair treated with conventional dyeing or lifting compositions.

As used herein, the process and composition disclosed herein may be used on the hair that has not been artificially dyed or pigmented.

As used herein, the process and composition disclosed herein may be also used on the hair that has been artificially dyed or pigmented.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The examples that follow serve to illustrate embodiments of the present disclosure without, however, being limiting in nature.

### Examples

The ingredient amounts in the compositions/formulas described below are expressed in % by weight, based on the total weight of the composition/formula.

### Example I

### Formulation Examples

TABLE 1

| | Formula A | Formula B | Formula C |
|---|---|---|---|
| | % by | % by | % by |
| INCI US/ingredient | weight | weight | weight |
| AMMONIUM CHLORIDE | 5.0 | 5.0 | — |
| ERYTHORBIC ACID | 0.1 | 0.1 | 0.1 |
| ETHANOLAMINE | 1.0 | 1.0 | 1.0 |
| EDTA | 0.2 | 0.2 | 0.2 |
| PROPYLENE GLYCOL | — | — | 5.0 |
| SODIUM SULFITE | 0.025 | 0.025 | 0.025 |
| MINERAL OIL | 60 | 60 | 60 |
| SODIUM ACETATE | — | — | 7.5 |
| ACRYLATES COPOLYMER | 5.5 | 5.5 | 4.0 |
| (Carbopol ® Aqua SF-1, | | | |
| Lubrizol, 30% activity | | | |
| in water) | | | |
| GLYCERIN | 5.0 | 5.0 | — |
| STEARETH-20 | 4.0 | — | — |
| PPG-5-CETETH-20 | — | 5.0 | — |
| WATER | QS 100 | QS 100 | QS 100 |

The formulas above were prepared as follow (1000 grams in total):

Acrylates Copolymer was added to water and the resulting solution was mixed using a VMI Rayneri mixer equipped with a chopper blade. 10 g of Ethanolamine was added to neutralize the acrylates copolymer (commercially available as Carbopol® Aqua SF-1). The solution became a clear thick gel upon complete neutralization. The speed of mixing was increased to at least about 600 rpm. The solution became thicker & turned into a white paste. 80 g of mineral oil was added to the solution to create an emulsion and the speed of mixing was increased to at least about 1000 RPM. One half of the remainder of the oil, was then added to the emulsion and mixing was continued for about 10 minutes. The speed of mixing was increased to at least about 1200 RPM to ensure proper mixing. One half of the total concentration of organic solvent (glycerin) was added to adjust or decrease the viscosity of the emulsion The remainder of the mineral oil was then added and mixing was continued for a few minutes. The salt was added as well. Any desired additional ingredients such as dyes or colorant compounds, chelants, an additional fatty substance and/or additives or auxiliary ingredients, were added at this stage. The anti-oxidants, sodium sulfite and erythorbic acid, were also added to the emulsion. The speed of mixing was decreased. The remainder of the ethanolamine was added to the emulsion to adjust the pH. Citric acid was used to adjust the pH, if necessary.

TABLE 2
Oxidizing Composition (to be mixed with each of the
inventive cosmetic compositions, Formulas A, B, and C)

| | Formula I (20 |
|---|---|
| | volume)% by |
| INCI US/Ingredients | weight |
| HYDROGEN PEROXIDE (50% activity in water) | 12 |
| MINERAL OIL | 20 |
| CETEARYL ALCOHOL AND STEARETH-20 | 11 |
| CATIONIC AGENTS - HEXADIMETHRINE | 0.75 |
| CHLORIDE, POLYQUATERNIUM-6 | |
| SODIUM STANNATE, PENTASODIUM | 0.22 |
| PENTETATE, TETRASODIUM PYROPHOSPHATE | |
| GLYCERIN | 0.5 |
| PHOSPHORIC ACID | pH adjuster |
| WATER | QS 100 |

TABLE 3
Comparative Cosmetic Compositions
(conventional/commercial formulas)

| Comparative | |
|---|---|
| cosmetic | |
| formulas | Key Ingredients |
| Formula D | Ethanolamine (4.2%), nonionic surfactants |
| (alkaline | (33%), carbomer (0.4%), cationic agents (7%), |
| system)* | glycol distearate (2%), propylene glycol (10%), |
| | lauric acid, silica dimethyl silylate, |
| | pentasodium pentetate, sodium metabisulfite, |
| | erythorbic acid, mica/titanium dioxide, apricot |
| | kernel oil and peach kernel oil (0.2%), water |
| Formula E | Ethanolamine (0.2%), nonionic surfactants |
| (acidic | (17%), cetyl hydroxyethyl cellulose (0.4%), |
| system)* | cationic agents (2.5%), glycerin (3%), sodium |
| | cetearyl sulfate (1.5%), trideceth-2 |
| | carboxaminde MEA (4%), sodium sulfite, |
| | erythorbic acid, mica/titanium dioxide, punica |
| | gratum seed oil (0.2%), EDTA, water |
| Formula F | Ethanolamine (4.2%), nonionic surfactants |
| (alkaline | (32.5%), carbomer (0.4%), cationic agents |
| system)* | (7.5%), glycol distearate (2%), propylene |
| | glycol (10%), lauric acid, sodium metasilicate, |
| | silica dimethyl silylate, pentasodium |
| | pentetate, sodium metabisulfite, erythorbic |
| | acid, punica gratum seed oil (0.2%), water |

*final composition when the formula is mixed with an oxidizing composition is either alkaline or acidic

TABLE 4
Oxidizing Compositions (to be mixed with comparative
cosmetic compositions, Formulas D, E, and F)

| | Formula II | Formula III |
|---|---|---|
| | (20 volume) | (10 volume) |
| INCI US | % by weight | % by weight |
| HYDROGEN PEROXIDE | 12 | 6 |
| (50% activity in water) | | |

Other ingredients for each formula: water, glycerin, cetearyl alcohol, ceteareth-25, pentasodium pentetate, tetrasodium pyrophosphate, trideceth-2 carboxaminde MEA, sodium stannate

Each of Formulas E and F was mixed with Formula II. Formula D was mixed with Formula III.

### Example II

### Colorimetric Measurements

Each of the inventive and comparative cosmetic compositions in Example I was mixed with the indicated oxidizing compositions and the resulting mixtures of compositions were then used on hair according to the following general procedure:
10 g of the base composition was mixed with 10 g of the oxidizing composition (1:1 ratio);
the resulting mixture or composition was applied onto hair swatches and left to stand on the hair for about 20 minutes;
the hair swatches were then washed with shampoo, rinsed and then dried.

If desired, the cosmetic composition can be mixed with the oxidizing composition in a 1:2 ratio or a 1:3 ratio or a 1:4 ratio. Unless specified otherwise, all ratios of cosmetic composition to oxidizing composition in the examples presented are 1:1 ratios.

The resulting mixtures or compositions of the invention had pH values of less than 7. The resulting mixtures or compositions obtained from comparative Formula D, E and F had pH values of 9.15, 6.75, and 9.54, respectively.

It was found that before mixing with an oxidizing composition, the inventive cosmetic compositions had an excellent, non-drip consistency. This consistency remained even after the inventive cosmetic compositions were mixed with the oxidizing compositions.

TABLE 5
Summary of pH and viscosity

| pH and Viscosity | Formula A | Formula B | Formula C |
|---|---|---|---|
| pH of cosmetic composition | 8.15 | 7.59 | 7.78 |
| pH of composition resulting | 6.69 | 6.57 | 6.63 |
| from mixing cosmetic | | | |
| composition and oxidizing | | | |
| composition | | | |
| Viscosity of cosmetic | 71.15 uD | 76.35 uD | 72.38 uD |
| composition (creamy texture) | | | |
| Viscosity of composition | 65.15 uD | 68.15 uD | 63.21 uD |
| resulting from mixing | | | |
| cosmetic composition and | | | |
| oxidizing composition | | | |
| (creamy texture) | | | |

For measuring the degree of change in the color of hair (e.g. degree of lightening/lifting color or color deposit) after treating the hair, the color of each swatch was measured with a Minolta CM2600d spectrocolorimeter (specular components included, 10 degrees angle, illuminant D65) in the CIEL*a*b* system.

Two parameters, L and ΔE (delta-E), were measured. L* represents the intensity of the color, a* indicates the green/red color axis and b* the blue/yellow color axis.

According to this system, the greater the value of L, the lighter or less intense the color. Conversely, the lower the value of L, the darker or more intense the color (this can also indicate greater color deposit when the composition contains colorants).

The ΔL or the difference between the L value for the treated hair versus the L value for the control hair swatch represents a change in the value of L: the more negative the ΔL value is, the darker the color that is deposited on the hair: ΔL=Lt (treated hair)−Lc (control hair)

Delta-E (ΔE) represents color change. If ΔE is less than 1.0 there is hardly any color difference that the human eye can see. If ΔE greater than 1.0, then there is a noticeable color difference.

The following tables show the color changes imparted to the hair.

TABLE 6
Colorimetric results

| Initial Hair | | Oxidizing | | |
|---|---|---|---|---|
| color (on 90% | Formula | Composition | | |
| gray hair) | (Cosmetic Composition) | (1:1 ratio) | L | ΔE |
| Natural | Control | | 21.02 | — |
| Level 3 | (untreated swatch) | | | |
| Hair* | Inventive Formula B | Formula I | 23.94 | 4.98 |
| | Comparative Formula E | Formula II | 22.42 | 3.53 |
| | Comparative Formula F | Formula II | 24.28 | 5.52 |
| Natural | Control | | 29.85 | — |
| Level 6 | (untreated swatch) | | | |
| Hair* | Inventive Formula B | Formula I | 37.58 | 4.58 |
| | Comparative Formula E | Formula II | 35.09 | 3.49 |
| | Comparative Formula F | Formula II | 36.21 | 3.54 |

*level 3 hair type is dark brown shade and level 6 hair type is medium brown shade.

TABLE 7
Colorimetric results

| Initial Hair | | | |
|---|---|---|---|
| color (on 90% | Formula | Oxidizing | |
| gray hair) | (Cosmetic Composition) | Composition | L |
| Natural | Control | | 19.44 |
| Level 3 | (untreated swatch) | | |
| Hair* | Inventive Formula A | Formula I | 22.94 |
| | Comparative Formula D | Formula III | 20.15 |
| | Comparative Formula E | Formula II | 21.42 |
| | Comparative Formula F | Formula II | 26.19 |
| Natural | Control | | 30.32 |
| Level 6 | (untreated swatch) | | |
| Hair* | Inventive Formula A | Formula I | 37.98 |
| | Comparative Formula D | Formula III | 33.96 |
| | Comparative Formula E | Formula II | 32.48 |
| | Comparative Formula F | Formula II | 42.21 |

*level 3 hair type is dark brown shade and level 6 hair type is medium brown shade.

The results above show that the color of the level 3 and level 6 hair treated in accordance with the invention was significantly lifted (i.e., lighter) as evidenced by the higher L values compared to the control hair swatch (observation by eye and colorimetric measurements). In addition, the degrees of lightening/lift and color changes on hair treated with the composition prepared from the cosmetic composition of the invention were comparable to those obtained with the compositions prepared from the comparative compositions (observation by eye and colorimetric measurement of delta). Thus, even at an acidic pH and with minimal amount of neutralizing agents (such as remaining or residual unreacted ethanolamine after neutralizing the acrylic polymer), the compositions of the invention were still able to lighten or lift the color of the hair as effectively as or even better compared to the comparative compositions an acidic composition prepared from Formula E and alkaline compositions prepared from Formulas D and F, each containing a significant amount of ethanolamine at 4.2% by weight.

### Example III

### Stability Studies

The inventive compositions, Formula A, B, and C above were heated from between 20° C.-60° C. in a controlled environment chamber. The compositions showed no phase separation and maintained their cream-like structure. Stability was also independent of the presence of oxidation dyes and levels of the high viscosity oil.

The stability of these formulas were also tested against a formula that did not contain salt e.g., ammonium chloride, sodium acetate. It was found that while the formula without the salt remained stable for 8 weeks, the inventive formulas remained stable for a longer period of time.

### Example IV

### Levels of Fatty Substance

TABLE 8
Inventive cosmetic compositions using various levels
of the fatty substance, mineral oil, are presented below

| | Control | Inventive Cosmetic compositions* | | | | | | |
|---|---|---|---|---|---|---|---|---|
| Raw Materials | formula | Formula B | Formula BB | Formula BC | Formula BD | Formula BE | Formula BF | Formula BG |
| ACRYLATES | 5.5 | 5.5 | 5.5 | 5.5 | 5.5 | 5.5 | 5.5 | 5.5 |
| COPOLYMER | | | | | | | | |
| (Carbopol ® Aqua | | | | | | | | |
| SF-1, Lubrizol, | | | | | | | | |
| 30% activity in | | | | | | | | |
| water) | | | | | | | | |
| EDTA | 0.2 | 0.2 | 0.2 | 0.2 | 0.2 | 0.2 | 0.2 | 0.2 |
| Monoethanolamine | 1.0 | 1.0 | 1.0 | 1.0 | 1.0 | 1.0 | 1.0 | 1.0 |
| Mineral Oil | 0 | 60 | 55 | 50 | 45 | 30 | 35 | 30 |
| Glycerin | 5.0 | 5.0 | 5.0 | 5.0 | 5.0 | 5.0 | 5.0 | 5.0 |
| Ammonium | 5.0 | 5.0 | 5.0 | 5.0 | 5.0 | 5.0 | 5.0 | 5.0 |
| Chloride | | | | | | | | |
| PPG-5-CETETH-20 | 5.0 | 5.0 | 5.0 | 5.0 | 5.0 | 5.0 | 5.0 | 5.0 |
| Sodium Sulfite | 0.025 | 0.025 | 0.025 | 0.025 | 0.025 | 0.025 | 0.025 | 0.025 |
| Erythorbic Acid | 0.1 | 0.1 | 0.1 | 0.1 | 0.1 | 0.1 | 0.1 | 0.1 |
| Deionized Water | QS 100 | QS 100 | QS 100 | QS 100 | QS 100 | QS 100 | QS 100 | QS 100 |

pH and Viscosity

| | | | | | | | | |
|---|---|---|---|---|---|---|---|---|
| pH of cosmetic | 8.67 | 8.69 | 8.72 | 8.74 | 8.55 | 8.63 | 8.14 | 7.98 |
| compositions | | | | | | | | |
| Viscosity of | 56.35 | 76.70 | 71.1 | 69.18 | 69.42 | 67.83 | 71.15 | 71.15 |
| cosmetic | | | | | | | | |
| compositions | | | | | | | | |
| (in Ud) | | | | | | | | |
| pH of | 6.67 | 6.56 | 6.5 | 6.58 | 6.66 | 6.76 | 6 .84 | 6.56 |
| composition | | | | | | | | |
| resulting from | | | | | | | | |
| mixing cosmetic | | | | | | | | |
| composition and | | | | | | | | |
| oxidizing | | | | | | | | |
| composition | | | | | | | | |
| Viscosity of | 61.15 | 76.35 | 71.15 | 71.15 | 71.15 | 71.15 | 71.15 | 71.15 |
| composition | | | | | | | | |
| resulting from | | | | | | | | |
| mixing cosmetic | | | | | | | | |
| composition and | | | | | | | | |
| oxidizing | | | | | | | | |
| composition | | | | | | | | |
| (in Ud) | | | | | | | | |
| L values for | 17.25 | 24.33 | 20.94 | 20.31 | 19.88 | 19.55 | 19.20 | 19.00 |
| Level 3 hair | | | | | | | | |
| swatches | | | | | | | | |
| L values for | 30.18 | 37.83 | 36.36 | 36.51 | 35.09 | 34.15 | 33.36 | 32.51 |
| Level 3 hair | | | | | | | | |
| swatches | | | | | | | | |

*each inventive composition was mixed with oxidizing composition (Formula I)

It was observed that the inventive cosmetic compositions remained stable even at very high concentrations of the high viscosity oil and that the viscosities remained the same or changed minimally even after they were mixed with the oxidizing composition.

The L values in the table above also show that the color of the hair was significantly more lifted or lighter after contacting the hair with the compositions prepared from the inventive cosmetic compositions as compared to the color of the hair contacted with the control formula. Moreover, the degree of lifting or lightening of the color increased with increasing amounts of fatty substance (mineral oil).

### Example V

### Levels and Degrees of Lift

### A. Inventive Formula B Versus Comparative Formula F

Half-head studies on the hair of human volunteers were conducted in order to compare the degrees of lift of color on hair provided by the compositions prepared from the inventive cosmetic compositions with those provided by the compositions prepared from the comparative cosmetic compositions composition. Processing time of these compositions (“test compositions”) on the hair was 20 minutes. The hair was then rinsed and dried.

The degree of lift in the color of the hair was determined by visually comparing the tone or lightness/darkness of the hair before and after contacting the hair with the test compositions (invention and comparative) and also by comparing the hair contacted with the compositions of the invention with the color tones of standard hair swatches.

TABLE 9
Comparison to an alkaline system

| | Natural | Level after treating hair | |
|---|---|---|---|
| | Level | Invention | Comparative |
| | before | Cosmetic composition, | Cosmetic composition, |
| | treating | Formula B + oxidizing | Formula F + oxidizing |
| Test | hair | composition, Formula I | composition, Formula II |
| 1 | 5.0 | 6.0 | 6.5 |
| 2 | 6.0 | 7.0 | 7.75 |
| 3 | 4.0 | 5.0 | 6.5 |

Formula B that did not contain salt (“formula B without salt”) was also tested on hair. The degree of color lift provided by formula B without salt ranged from zero up to 0.25 (for example, if the starting color level of untreated hair was 5, after treating the hair with formula B without salt, the color level was at from between 5 to 5.25).

The results in Table 9 above surprisingly and unexpectedly showed that the composition of the invention consistently lifted the color of the hair by one level, i.e., the degree of lift was 1. This color lifting capability of the inventive composition allows for enhanced gray coverage when dye compounds are present in the inventive cosmetic composition. While Formula F demonstrated greater lift, the results for this comparative formula can be attributed to its alkaline nature due to the presence of higher levels of ethanolamine (4.2% by weight) which is less desirable with respect to the condition of or adverse effect on the hair fibers. On the other hand, the composition prepared from the cosmetic composition of the invention still produced an acceptable level of lift to the color of the hair even when the amount of ethanolamine in the base formula was much lower at about 1% or less by weight such that the composition had a pH below 7 (acidic system).

### Example VI

### Inventive Cosmetic Formulation Examples with Dyes

TABLE 10
Formula A with dyes

| | Dark | Garnet | Polished | | Cool | |
|---|---|---|---|---|---|---|
| Raw Materials | Chocolate | Sand | Ruby | Iced Gold | Cocoa | Bonfire |
| ACRYLATES | 5.50 | 5.50 | 5.50 | 5.50 | 5.50 | 5.50 |
| COPOLYMER | | | | | | |
| (Carbopol ® Aqua | | | | | | |
| SF-1, Lubrizol, | | | | | | |
| 30% activity in | | | | | | |
| water) | | | | | | |
| EDTA | 0.20 | 0.20 | 0.20 | 0.20 | 0.20 | 0.20 |
| Monoethanolamine | 1.00 | 1.00 | 1.00 | 1.00 | 1.00 | 1.00 |
| Mineral Oil | 60.00 | 60.00 | 60.00 | 60.00 | 60.00 | 60.00 |
| Glycerin | 5.00 | 5.00 | 5.00 | 5.00 | 5.00 | 5.00 |
| Ammonium Chloride | 5.00 | 5.00 | 5.00 | 5.00 | 5.00 | 5.00 |
| Stearth-20 | 4.00 | 4.00 | 4.00 | 4.00 | 4.00 | 4.00 |
| Sodium Sulfite | 0.03 | 0.03 | 0.03 | 0.03 | 0.03 | 0.03 |
| Erythorbic Acid | 0.10 | 0.10 | 0.10 | 0.10 | 0.10 | 0.10 |
| Dye Concentration - | 1.256 | 1.1 | 2.48 | 1.02 | 0.48 | 1.908 |
| Oxidative & | | | | | | |
| Direct Dyes* | | | | | | |
| Deionized Water | QS 100 | QS 100 | QS 100 | QS 100 | QS 100 | QS 100 |

pH and Viscosity

| | | | | | | |
|---|---|---|---|---|---|---|
| pH of cosmetic | 8.00 | 7.15 | 7.67 | 7.74 | 7.67 | 7.71 |
| compositions | | | | | | |
| pH of composition | 6.71 | 6.67 | 6.59 | 6.62 | 6.59 | 6.53 |
| resulting from | | | | | | |
| mixing cosmetic | | | | | | |
| composition and | | | | | | |
| oxidizing | | | | | | |
| composition | | | | | | |
| Viscosity of | 59.60 | 72.35 | 73.60 | 69.99 | 73.62 | 63.11 |
| cosmetic | | | | | | |
| compositions | | | | | | |
| (in uD) | | | | | | |
| viscosity of | 43.22 | 69.28 | 65.49 | 59.51 | 65.49 | 58.62 |
| composition | | | | | | |
| resulting from | | | | | | |
| mixing cosmetic | | | | | | |
| composition and | | | | | | |
| oxidizing | | | | | | |
| composition | | | | | | |
| (in UD) | | | | | | |

*total amount of dyes is less than conventional/typical dyeing compositions

### Example VII

### Colorimetric Measurements

Compositions for application onto hair swatches were prepared from the formulas in Table 10 above and their corresponding oxidizing composition. These prepared compositions were applied onto hair swatches according to the procedure described in Example II. Colorimetric measurements of the hair contacted with the compositions were taken.

TABLE 11
L values

| | | | | | | |
|---|---|---|---|---|---|---|
| | Dark | Garnet | Polished | | | |
| Characteristics | Chocolate | Sand | Ruby | Iced Gold | Cool Cocoa | Bonfire |

L values; Unpermed hair - nonchemically treated hair

| | | | | | | | |
|---|---|---|---|---|---|---|---|
| Unpermed - | 61.88 | | | | | | |
| Untreated | | | | | | | |
| hair | | | | | | | |
| Formula E | | 22.1 | 39.2 | 41.6 | 49.6 | 27.74 | |
| with dyes | | | | | | | |
| Formula A | | 19.6 | 36.5 | 38.6 | 45.7 | 25.33 | 26.6 |
| with dyes | | | | | | | |
| (invention) | | | | | | | |
| Formula F | | | | | | | 28.6 |
| with dyes | | | | | | | |

L values; Permed hair - Chemically treated hair

| | | | | | | | |
|---|---|---|---|---|---|---|---|
| Permed - | 60.92 | | | | | | |
| Untreated | | | | | | | |
| Formula E | | 21.9 | 37.1 | 40.1 | 48.6 | 25.69 | |
| with dyes | | | | | | | |
| Formula A | | 18.73 | 35.69 | 36.48 | 43.21 | 23.17 | 28.36 |
| with dyes | | | | | | | |
| (invention) | | | | | | | |
| Formula F | | | | | | | 26.12 |
| with dyes | | | | | | | |

The compositions prepared from the inventive Formula A imparted excellent color deposit as evidenced by the significantly lower L values compared to the L values for untreated hair (both permed and unpermed). At the same time, using the same dye concentrations, the hair contacted with the compositions prepared from inventive Formula A exhibited comparable and/or greater color deposit as evidenced by the L values compared to the hair contacted with the compositions prepared from the comparative Formulas.

### Example VIII

### Levels and Degrees of Lift Using Formulas with Dyes

Half-head studies on the hair of human volunteers were conducted in order to determine the degrees of lift of color on hair provided by the compositions prepared from the inventive cosmetic compositions. Processing time of these compositions on the hair was 20 minutes. The hair was then rinsed and dried. The level of color or tone height of the hair was determined as described above.

TABLE 12

| | | Level after treating |
|---|---|---|
| | | hair |
| | | Invention |
| | Natural Level before | Formula B with dyes + |
| Test | treating hair | Formula I |
| 1 | 5.0 | 5.75 |
| 25 to 50% gray | | |
| hair | | |
| 2 | 5.0 | 5.5 |
| 50 to 75% gray | | |
| hair | | |
| 3 | 5.0 | 5.5 |
| 100% gray hair | | |

The results above surprisingly and unexpectedly showed that the composition of the invention consistently lifted the color of the hair by at least 0.5 level when the cosmetic composition contained dyes. Thus, the inventive composition imparts lift to color of hair (increase in tone height or color level) while allowing for enhanced gray coverage in the presence of dye compounds.

### Example IX

### Hair Damage

The extent of hair damage (if any) imparted to hair by contacting the hair with the test inventive and test comparative conventional acidic and alkaline compositions was evaluated based on the texture and feel of the hair and by determining the amount of cysteic acid in the hair using amino acid analysis. The cysteic acid content is one way to measure the amount of damage to hair fibers caused by various chemical treatments performed on hair such as dyeing, lightening and bleaching. The higher the cysteic acid content, the more damage done to the hair. The cysteic acid content can be correlate to the degrees of damage imparted to the hair.

Hair samples were processed with 1 cycle (1× application) or 5 cycles of contacting the hair with the product, leaving the product for a period of time on the hair, washing the hair, and then drying the hair

It was observed that the hair contacted with the inventive composition, formula A, and compositions prepared from the acidic comparative composition had a smoother feel than the hair contacted with the alkaline comparative compositions.

TABLE 13

Level of cysteic acid

| Treatment on Hair samples | 1X application | 5X application |
|---|---|---|
| Control - untreated | 0.5 | 0.5 |

Treatment product: Cosmetic composition + oxidizing composition

| | | |
|---|---|---|
| Formula A (Invention) + | 0.7 | 0.9 |
| Formula II | | |
| Comparative composition, | 0.7 | 0.8 |
| acidic system | | |
| Comparative composition, | 0.8-1.0 | 1.4-2.5 |
| alkaline system | | |

Based on the measured amounts of cysteic acid, the degree of damage to the hair contacted with the composition prepared from the inventive formula A was: (i) comparable to that assessed from the hair treated with the acidic comparative composition at 1× and 5× applications; and (ii) significantly less than the degree of damage assessed from the hair treated with the alkaline comparative composition.

These results show that the compositions of the invention in an acidic system are able to lift the color of hair and deposit color effectively onto hair while leaving the hair less damaged, even when these compositions contained less amount of dyes than typical hair dyeing compositions.

### Example X

### Evaluating Lift with Different Salts

TABLE 14

| | | pH | Observed |
|---|---|---|---|
| | | (cosmetic | levels of |
| | concentration | composition/ | lift on hair |
| Salt | (% by wt) | mix) | swatches |
| Ammonium sulfate | 7.5 | 8.52/6.53 | 1-2 |
| Sodium sulfate | 5.0 | 8.75/6.68 | 0.5-1 |
| Manganese gluconate | 5.0 | 8.12/6.74 | 0.5-2 |
| Calcium chloride | 5.0 | 7.59/6.71 | 0.5-1 |
| Agmatine sulfate | 7.5 | 7.12/6.49 | 0.5-2 |
| Sodium acetate | 7.5 | 8.72/6.56 | 0.5-2 |
| Ammonium chloride | 5.0 | 8.69/6.78 | 2 |

It will be apparent to those skilled in the art that various modifications and variations can be made in the delivery system, composition and methods of the invention without departing from the spirit or scope of the invention. Thus, it is intended that the present invention cover the modifications and variations of this invention provided that they come within the scope of the appended claims and their equivalents.

What is claimed is:

1. A cosmetic composition for keratin fibers comprising:
(a) from about 10% to about 80% by weight of at least one fatty substance;
(b) at least one acrylic polymer selected from crosslinked copolymers of (meth)acrylic acid and/or (C1-C6)alkyl esters and from acrylic associative polymers;
(c) from about 0.5% to about 15% by weight of at least one salt selected from an ammonium salt other than an ammonium acetate salt, a quaternary ammonium salt, a quaternary diammonium salt, an alkaline earth metal salt, a transition metal salt, an alkali metal salt other than an alkali metal phosphate salt, an agmatine salt, and mixtures thereof; and
(d) at least one cosmetically acceptable solvent;
all weights being based on the total weight of the composition.

2. The cosmetic composition according to claim 1, wherein the fatty substance is selected from alkanes, fatty alcohols, esters of fatty acid, esters of fatty alcohol, hydrocarbons, silicones, non-silicone oils selected from mineral, vegetable and synthetic oils, non-silicone waxes, and mixtures thereof.

3. The cosmetic composition according to claim 2, wherein the fatty substance is present in an amount of from about 15% to about 70% by weight, based on the total weight of the composition.

4. The cosmetic composition according to claim 3, wherein the fatty substance is present in an amount of from about 30% to about 60% by weight, based on the total weight of the composition.

5. The cosmetic composition according to claim 4, wherein the acrylic polymer is present in an amount of from about 0.1% to about 10% by weight of active material, based on the total weight of the composition.

6. The cosmetic composition according to claim 5, wherein the acrylic polymer is selected from a crosslinked (meth)acrylic acid/ethyl acrylate copolymer, a cross-linked anionic acrylate polymer, and mixtures thereof.

7. The cosmetic composition according to claim 6, wherein the crosslinked (meth)acrylic acid/ethyl acrylate copolymer is an acrylates copolymer in the form of an aqueous dispersion.

8. The cosmetic composition according to claim 6, wherein the cross-linked anionic acrylate polymer is acrylates crosspolymer-4.

9. The cosmetic composition according to claim 5, wherein the acrylic polymer is selected from acrylic associative polymers acrylic comprising anionic amphiphilic polymers.

10. The cosmetic composition according to claim 5, further comprising from about 0.01% to less than about 5% by weight of at least one neutralizing agent other than (c) and selected from alkali metal carbonates, alkali metal phosphates, organic amines, hydroxide base compounds, and mixtures thereof.

11. The cosmetic composition according to claim 5, wherein the salt is present in an amount of from about 1% to about 10% by weight, based on the total weight of composition.

12. The cosmetic composition according to claim 11, wherein the salt is an ammonium salt selected from ammonium carbonate, ammonium carbamate, ammonium hydrogen carbonate, ammonium sulfate, ammonium glycerophosphates, ammonium chloride, ammonium nitrate, and mixtures thereof.

13. The cosmetic composition according to claim 12, wherein the ammonium salt is ammonium chloride.

14. The cosmetic composition according to claim 11, wherein the salt is selected from an alkaline earth metal salt, a transition metal salt, an alkali metal salt other than an alkali metal phosphate salt, and an agmatine salt.

15. The cosmetic composition according to claim 14, wherein the salt is selected from calcium chloride, manganese gluconate, sodium acetate, sodium sulfate, agmatine sulfate, and mixtures thereof.

16. The cosmetic composition according to claim 1, wherein the cosmetically acceptable solvent is selected from water and a water/organic solvent mixture.

17. The cosmetic composition according to claim 11, wherein the composition further comprises at least one oxidizing agent selected from peroxides, urea peroxide, alkali metal bromates, ferricyanides, peroxygenated salts, perborates, percarbonates, laccases, peroxidases, redox enzymes, and mixtures thereof.

18. The cosmetic composition according to claim 17, wherein the pH of the composition ranges from about 2 to about 7.

19. The cosmetic composition according to claim 18, wherein the pH of the composition ranges from about 4 to about 6.9.

20. The cosmetic composition according to claim 1, further comprising at least one colorant compound selected from oxidative dye precursors, direct dyes, pigments, and mixtures thereof.

21. The cosmetic composition according to claim 20, further comprising at least one nonionic surfactant other than the fatty substance in (a).

22. The cosmetic composition according to claim 17, wherein the composition is substantially free of ammonia.

23. The cosmetic composition according to claim 1, wherein the composition is capable of being mixed with an oxidizing composition comprising at least one oxidizing agent selected from peroxides, urea peroxide, alkali metal bromates, ferricyanides, peroxygenated salts, perborates, percarbonates, laccases, peroxidases, redox enzymes, and mixtures thereof, and a cosmetically acceptable solvent selected from water and a water/organic solvent mixture.

24. A composition for altering the color of hair comprising:
A. a cosmetic composition containing:
(a) from about 30% to about 60% by weight of at least one fatty substance;
(b) from about 1.2% to about 1.65% by weight of active material of an acrylic polymer selected from a crosslinked (meth)acrylic acid/ethyl acrylate copolymer, acrylates crosspolymer-4, and mixtures thereof;
(c) from about 5% to about 7.5% by weight of at least one salt selected from ammonium chloride, ammonium sulfate, calcium chloride, manganese gluconate, sodium acetate, sodium sulfate, agmatine sulfate, and mixtures thereof; and
(d) at least one cosmetically acceptable solvent;
all weights being based on the total weight of the composition;
B. an oxidizing composition containing at least one oxidizing agent selected from peroxides, urea peroxide, alkali metal bromates, ferricyanides, peroxygenated salts, perborates, percarbonates, laccases, peroxidases, redox enzymes, and mixtures thereof, and a cosmetically acceptable solvent selected from water and a water/organic solvent mixture;
wherein the pH of the composition for altering the color of hair ranges from about 2 to about 7.

25. The composition for altering the color of hair of claim 24, wherein the cosmetic composition further comprises: (i) at least one colorant compound selected from oxidative dye precursors, direct dyes, pigments, and mixtures thereof and (ii) at least one nonionic surfactant.

26. The composition for altering the color of hair of claim 25, wherein the acrylic polymer is an acrylates copolymer in the form of an aqueous dispersion.

27. A composition for altering the color of hair comprising:
(a) from about 5% to about 60% by weight of at least one fatty substance;
(b) from about 0.05% to about 5% by weight of active material of an acrylic polymer selected from crosslinked copolymers of (meth)acrylic acid and/or (C1-C6)alkyl esters and from acrylic associative polymers;
(c) from about 0.25% to about 7.5% by weight of at least one salt selected from an ammonium salt other than an ammonium acetate salt, a quaternary ammonium salt, a quaternary diammonium salt, an alkaline earth metal salt, a transition metal salt, an alkali metal salt other than an alkali metal phosphate salt, an agmatine salt, and mixtures thereof;
(d) at least one cosmetically acceptable solvent; and
(e) from about 0.01 to about 1% by weight of at least one neutralizing agent other than (c) and selected from alkali metal carbonates, alkali metal phosphates, organic amines, hydroxide base compounds, and mixtures thereof;
(f) at least one colorant compound selected from oxidative dye precursors, direct dyes, pigments, and mixtures thereof;
(g) at least one nonionic surfactant selected from alkoxylated nonionic surfactants; and
(h) at least one oxidizing agent;
all weights being based on the total weight of the composition; and
wherein the pH of the composition ranges from about 4 to about 6.9.

28. A process for altering the color of hair, comprising contacting hair with a composition for lifting the color of hair for a sufficient period of time to achieve a desired level of lift of the color of the hair; wherein the composition is formed from mixing the cosmetic composition of claim 1 with an oxidizing composition containing at least one oxidizing agent and a cosmetically acceptable solvent selected from water and a water/organic solvent mixture; and wherein the pH of the composition for altering the color of hair ranges from about 1 to about 7.

29. The process according to claim 28, further comprising leaving the composition on the hair for a time period sufficient to achieve an increase of 0.5 to 4 in the tone height of the hair.

30. The process according to claim 28, further comprising leaving the composition on the hair for a time period of up to about 60 minutes.

31. The process according to claim 28, further comprising leaving the composition on the hair for a time period of from about 5 minutes to about 20 minutes.

32. The process according to claim 28, wherein the cosmetic composition further comprises: (i) at least one colorant compound selected from oxidative dye precursors, direct dyes, pigments, and mixtures thereof and (ii) at least one nonionic surfactant.

33. A multi-compartment kit for altering the color of hair comprising:
A. a first compartment containing a cosmetic composition comprising:
(a) from about 10% to about 80% by weight of at least one fatty substance;
(b) from about 0.1% to about 10% by weight of active material of at least one acrylic polymer selected from crosslinked copolymers of (meth)acrylic acid and/or (C1-C6)alkyl esters and from acrylic associative polymers;
(c) from about 0.5% to about 15% by weight of at least one salt selected from an ammonium salt other than an ammonium acetate salt, a quaternary ammonium salt, a quaternary diammonium salt, an alkaline earth metal salt, a transition metal salt, an alkali metal salt other than an alkali metal phosphate salt, an agmatine salt, and mixtures thereof; and
(d) at least one cosmetically acceptable solvent;
all weights being based on the total weight of the composition; and
B. a second compartment containing an oxidizing composition comprising at least one oxidizing agent selected from peroxides, urea peroxide, alkali metal bromates, ferricyanides, peroxygenated salts, perborates, percarbonates, laccases, peroxidases, redox enzymes, and mixtures thereof, and a cosmetically acceptable solvent selected from selected from water and a water/organic solvent mixture.

34. The multi-compartment kit according to claim 33, wherein the cosmetic composition further comprises: (i) at least one colorant compound selected from oxidative dye precursors, direct dyes, pigments, and mixtures thereof and (ii) at least one nonionic surfactant.
